(19) United States
(12) Patent Application Publication
(10) Pub. No.: US 2025/0388844 A1
(43) Pub. Date: Dec. 25, 2025

(54) ALGAE CULTIVATION AND HARVESTING FROM CARRIERS

(71) Applicant: ALGAFILM TECHNOLOGIES LTD., Ancaster (CA)

(72) Inventors: Pierre Lucien COTE, Ancaster (CA); Ahren BRITTON, North Vancouver (CA); Steven Kristian PEDERSEN, Nottawa (CA)

(21) Appl. No.: 19/243,540

(22) Filed: Jun. 19, 2025

Related U.S. Application Data
(60) Provisional application No. 63/662,154, filed on Jun. 20, 2024.
(60) Provisional application No. 63/672,033, filed on Jul. 16, 2024.

(51) Int. Cl. C12M 1/00; C12M 1/26

(52) U.S. Cl. CPC C12M 29/06; C12M 33/00

(57) ABSTRACT

Microalgae are grown in a biofilm on carriers. The carriers may have surfaces with a small angle to the vertical, optionally forming a cone. A surface of the carrier may be flexible and optionally include a porous material. Algae are harvested from the carriers using a water jet, which may provide a substantially solid stream of water. A nozzle for producing the water jet may spin while being moved laterally over a carrier bed. The water jet may dislodge algae from a surface of the carriers in the form of large agglomerates or blobs. The dislodged algae may have a high concentration of solids or readily settle. The algae may be collected from a floor below the carriers, optionally with a moving collection device. A moving harvesting device may treat a portion of a carrier bed such that harvesting of the entire bed is distributed over time.

### RELATED APPLICATION

This application claims the benefit of, and priority to, U.S. Provisional Application No. 63/662,154, filed on Jun. 20, 2024, and U.S. Provisional Application No. 63/672,033, filed on Jul. 16, 2024, both of which are incorporated herein by reference.

### FIELD

This specification relates to carriers, systems and methods useful for

growing algae and to systems and methods to harvest microalgae growing in a biofilm on carriers.

### BACKGROUND

Microalgae are photosynthetic organisms from multiple natural groups having a size up to a few hundred micrometers. Their elemental composition is represented by the Redfield ratio: C₁₀₆H₂₆₃O₁₁₀N₁₆P₁. Algae use inorganic carbon through photosynthesis as a source of carbon, and produce oxygen. On a mass basis, every kg of algae produced consumes about 2.0 kg of CO₂. Algae also need nutrients, for example nitrogen and phosphorus, which they extract from water in the form of ammonia, nitrate, or orthophosphate. Every kg of algae produced requires approximately 100 g of inorganic nitrogen and 10 g of orthophosphate (measured as phosphate).

Species of microalgae grown commercially include Chlorella, Spirulina, Dunaliella salina, Haematococcus pluvialis, Scenedesmus, Phormidium, Botryococcus, Chlamydomonas, etc.

The current global production of microalgae is about 20,000 dry tonnes per year. Two strains, Chlorella and Spirulina account for 90% of global production. These are processed for human food, extraction of nutraceuticals and animal food. A typical production rate for algae grown in suspension in water is in the range of 5-15 grams per day per meter square of reactor exposed to light (g/m²/d). CO₂ can be transferred from the air, but higher production rates are achieved by bubbling the water with a CO₂-rich gas. For these applications, commercial fertilizers are added as a source of nutrients.

There has been a large effort to grow algae for their energy content through conversion into biofuel. While research and development continue, production costs are still too high; a production target of 25 g/d/m² has been proposed to make algae cost effective to produce biofuels. Current efforts are focused on the production of sustainable aviation fuels.

More recently, there has been interest in growing algae to capture atmospheric CO₂ from the air to fight climate change. Useful products could be extracted from the algae before the residual carbon biomass is put away to sequester carbon.

Wastewater treatment is an emerging algae application. In addition to removing nitrogen and phosphorus that they need for growth, algae produce photosynthetic oxygen that can be used by bacteria in mixotrophic cultures to remove organic carbon (i.e., biological oxygen demand). Synergistically, the carbon dioxide produced by bacteria can be used as a source for algae growth.

The vast majority of commercial microalgal biomass is produced in ponds using natural light. Raceway ponds are shallow recirculating reactors where algae grow in suspension. The maximum concentration of suspended solids is circa 500 mg/L, limited by light penetration.

Raceway ponds occupy large areas of land. There have been efforts to intensify production by removing the rate limiting steps in the production of algae, such as algae concentration, carbon dioxide and nutrient concentration, surface area exposed to light, etc.

Algae process light through photosynthesis up to a flux of about 200 μmol photons/m²/s (26 kWh/m²/month). Above that, light saturation can be detrimental to growth. For example, in sunny climates such as the South of Spain, light flux can exceed this threshold by a factor of 5 in January and up to 10 in July.

Photobioreactors can be enclosed or exposed to atmosphere. Enclosed photobioreactors are preferred for high value-added products when it is beneficial to protect the algae from external contamination, or when using a gas source concentrated in CO₂. For other applications, reactors exposed to atmosphere may be simpler as they allow direct gas exchange.

U.S. Pat. No. 8,101,080 describes an enclosed photobioreactor consisting in a series of recirculating horizontal plastic tubes called tubular fence, filled with an algae suspension. The effluent is filtered through a membrane and a portion of the algae is returned to the reactor. Algae concentration in the reactor can reach 800-1,000 mg/L.

DE 10 2010 008 093 A1 describes an enclosed photobioreactor comprised of transparent hanging bags. The bags are deployed in rows with sufficient distance between rows to allow natural light to reach the algae suspension inside the bags. Algae can be further concentrated using a membrane filter.

U.S. Pat. No. 10,829,398 describes a membrane photobioreactor with light emitting diodes (LED) tubes immersed in the algal suspension.

As an alternative to suspended growth, microalgae can also be grown in a biofilm.

A rotating disk called AlgaDisk was developed in the framework of a European Union project for the purpose of carbon dioxide capture and biomass production. The disks are partially immersed and covered to control the gas atmosphere. A carbon dioxide rich gas is bubbled in the reactor. Another rotating biofilm support called the Algaewheel is described in U.S. Pat. No. 7,850,848. It is specific to wastewater treatment and involves floating wheels mounted on a shaft and positioned over aerators to impart a rotating movement.

U.S. Pat. Nos. 9,932,549 and 10,125,341 describe an algal biofilm growing on travelling flexible material mounted over rollers to circulate the algal population alternatively between an aqueous and a gaseous phase. The algae are harvested by scraping the biofilm off the travelling band.

EP 3360954A1 discloses growing algae attached to a floating fabric. Using a hydrophilic material for the fabric allows homogeneous and continuous wetting of the fabric.

U.S. Pat. No. 4,333,263, U.S. Pat. No. 4,496,096, U.S. Pat. No. 5,097,795, U.S. Pat. No. 5,572,770, U.S. Pat. No. 5,851,398, U.S. Pat. No. 8,375,627 and U.S. Pat. No. 20150189833 describe a system called the algal turf scrubber in which water flows over an inclined surface covered by a 3-dimensional screen that serves for benthic algae attachment. Algae are harvested by hydraulic or mechanical means.

U.S. Pat. No. 20,170,127656 describes vertical surfaces in the form of plates or cylinders hung over a water surface to support algae biofilm. Water is sprayed onto the carriers from above. A conveyor belt which can be water-permeable is located between the water surface and the carriers to collect algae that fall from the carriers and deposit the algae into a receptacle. Another approach using vertical surfaces is disclosed in WO 2015131830A1.

US2014/0127776 describe a vertical substrate system consisting of pieces

of cloth hung from a scaffolding system sprayed with an algae suspension and irrigated with water containing nutrients. The algae are harvested every 3-4 days using a mechanical roller press.

U.S. Pat. No. 8,895,279 describes a photobioreactor comprised of partially immersed rotating disks to alternatively expose a biofilm to a nutrient-laden liquid and an illuminated gas phase. The biofilm is harvested with doctor blades disposed adjacent to the growth disks.

US 2011/0217764 A1 and US 2013/0337548 A1 disclose an apparatus that exposes a biofilm growth surface to liquid media as it rotates. A rope is wound around a rotatable body capable of supporting biofilm growth. A harvester receives the biofilm laden rope, collects the biofilm by scraping and reloads the rope onto the rotatable body.

### SUMMARY

This specification describes a system and method that can be used to

grow algae, for example on a carrier or in a biofilm, or to harvest algae, for example algae growing on a carrier or in a biofilm, or to both grow and harvest algae. Algae can be grown, for example, to produce algae as product, to remove carbon dioxide from air, or to treat wastewater with the algae.

In some systems or methods, harvesting includes dislodging the algae from a carrier using water directed at the carrier, for example water in the form of a water jet. The water may be emitted from one or more nozzles and travel through air to reach the carrier.

In some systems or methods, a nozzle rotates about an axis of the nozzle, optionally in response to the emission of a water jet from the nozzle. The axis of the nozzle may be oblique to a water jet emitted from the nozzle. Water may be emitted downwards from the nozzle in a spray pattern within a cone, for example a cone having a half apex angle (alternatively called a semi-vertical angle) of 45 degrees or less, 30 degrees or less or 20 degrees or less. In some embodiments, rotating nozzles are capable of generating a solid water jet spinning at a rate of 50-500 rpm. Optionally, the rotating nozzles have an orifice ranging from 1-2 mm in inside diameter. A rotating nozzle may be supplied water at a pressure in the range of 2-15 bar or generate a flow rate of 1-10 l/min.

In some systems or methods, a carrier has a vertical or nearly vertical surface for growing algae. A plurality of carriers may be arranged in a pattern in plan view. One or more nozzles may be located above the carrier. An axis of the nozzles may be generally vertical. Water emitted from the nozzle may impact a surface of the carrier at an angle of 45 degrees or less, 30 degrees or less or 20 degrees or less.

In some systems or methods, a nozzle may translate, for example in a direction oblique to an axis of the nozzle. Optionally, a nozzle may both rotate and translate. The translation may be generally in a horizontal plane, optionally in two or more directions. Optionally, the translation has a velocity 0.5-35.0 m/min, for example 1-10 m/min. Optionally, translation of the nozzles has a primary direction that is oblique to the slope of a floor below one or more carriers.

In some systems or methods, a plurality of nozzles may translate

collectively. Water may be sprayed from the nozzles in patterns that overlap each other. Optionally, the nozzles are collectively mounted on a device, such as a cart, carriage or manifold, to facilitate moving the nozzles. Optionally, a spacing between nozzles and/or translation of a nozzle are such that a cone-shaped path of two water jets overlaps by 25-100%.

In some systems or methods, one or more nozzles may direct water at a portion of a carrier bed such that harvesting of the entire bed is distributed over time, for example 3-10 days. During this time, each carrier may receive water from the nozzles for only 1 hour or less, or 10 minutes or less. The volume of water used to dislodge the algae may be 2-10 L/m² of carrier surface area.

In some systems or methods, dislodged algae is collected on a floor below one or more carriers. The floor is optionally a sloped, for example towards a drain or collection channel. However, even if the floor is sloped, most (e.g. 50% or more) of dislodged algae may remain on the floor, for example because the dislodged algae does not flow to the drain or collection channel. Dislodged algae can be recovering from the floor, for example by vacuuming or by moving the algae across the floor by pushing, scraping or blowing. Optionally, the algae may be recovered using a moving collection device. Algae may be recovered from the floor in a slurry, or at a concentration of 1-50 g/L, 1-10 g/L, 5-50 g/L or 10-30 g/L. The recovered algae may be concentrated after collection, for example by settling.

In some systems or methods, a controller for a device to harvest algae controls one or more of the flow of water to a nozzle, the speed of movement of a nozzle, the path of movement of a nozzle, the frequency of harvesting, the timing of harvesting, the duration of harvesting, and the positioning of a nozzle.

In some systems or methods, a traveling bridge has an electrical, fluidic or mechanical drive to move and/or index the lateral position of a manifold or other device holding one or more nozzles between passes of the bridge across a carrier bed. The nozzles may achieve substantially full spray coverage of the carrier bed with multiple passes and one or more changes in the lateral position of the device. Optionally, a traveling bridge has a plurality of devices, the flow to each of which is optionally controlled by valves actuated by a control system.

In some systems or methods, an algae biofilm is grown on a carrier. Spraying the carrier with water dislodges at least some of the algae. Dislodged algae falls from the carrier to a floor below the carrier. Algae is recovered from the floor, optionally as a slurry. A recovery device may move on or over the floor to recover the algae. The algae may be recovered for example by vacuuming, pushing, blowing or scraping.

In some systems or methods, an algae growing plant (e.g. a plant to produce algae or to treat wastewater with algae) has one or more of a floor, algae carriers suspended over the floor, irrigation sprinklers suspended over the carriers, a moving bridge to move a harvesting nozzle over the carriers, and a recovery device that moves on or over the floor to recover dislodged algae. Growing algae can include growing an algae biofilm on a carrier, spraying the carrier to move the algae from the carrier to a floor and recovering algae from the floor.

Some systems or methods may include other combinations or sub-combinations of apparatus elements or process steps, or both, described in this specification.

### BRIEF DESCRIPTION OF THE FIGURES

FIG. 1 shows the cross section of examples of carriers that are symmetrical to a vertical plane (U, V or inverted V) or to a vertical axis (cone).

FIG. 2 is a plan view a 1.0 m² section of reactor covered with inverted V (FIG. 2a) or cones (FIG. 2b).

FIG. 3 a graph illustrating the surface area ratio of a pleated fabric and a cone.

FIG. 4a and FIG. 4b show examples of reactors with carriers in the

shape of a cone.

FIG. 5 is another example of a reactor with carriers in the shape of a cone.

FIG. 6a shows a reactor for growing algae in suspension in elevation view and FIG. 6b shows part of the reactor of FIG. 6a in plan view.

FIG. 7 shows a reactor for growing algae on a stationary carrier in the shape of a cone.

FIG. 8a shows a reactor for growing algae on suspended carriers in the shape of cones. FIG. 8b shows a carrier of FIG. 8a.

FIG. 9 is a graph showing the correlation between algae concentration expressed as turbidity and total suspended solids.

FIG. 10 is a microscope photo of algae from an algae biofilm grown in an experimental example.

FIG. 11 shows the results of algae grown in suspension.

FIG. 12 shows the conditions for inoculation of a carrier.

FIG. 13 are pictures of carriers before and after harvesting algae.

FIG. 14 shows the results of algae grown on suspended cones.

FIGS. 15A and 15B show plan views of the movement of a nozzle assembly over a carrier bed

FIGS. 16A and 16B show side views of a rotating nozzle illustrating overlap of the rotating nozzle path.

FIG. 17 is an isometric drawing of a pilot reactor used to grow algae.

FIGS. 18A and 18B show a nozzle assembly mounted on a traveling arm over a carrier bed of the reactor of FIG. 17.

FIG. 19 is a picture of the reactor floor of FIG. 17 showing agglomerates of algae during a harvest.

FIG. 20 is a composite of pictures of graduated cylinder taken at

different times showing rapid algae settling.

FIG. 21 shows an arrangement in plan view of irrigation sprinklers around a carrier bed.

FIG. 22 shows a plan view of an algae growing plant.

FIG. 23 shows section A′-A of the algae plant of FIG. 22 before installing sprinklers and carriers.

FIG. 24 shows section A′-A of the algae plant of FIG. 22 after installing sprinklers and carriers.

FIG. 25 shows an end view of section B-B′ of the algae plant of FIG. 22.

FIG. 26 shows an irrigation or collection carriage moving device for the algae plant of FIG. 22.

FIG. 27 shows an orthographic projection of a stripping cart.

FIG. 28 shows an orthographic projection of a recovery cart.

### DETAILED DESCRIPTION

The words “preferably”, “preferred” and similar words are used in this specification to refer to features that are optional and may be advantageous in at least some circumstances. The word “may” is also used to refer to something optional.

The words “spray” and “spraying” and similar words are used in this specification to include the possibility of, but not require, that the liquid be broken into drops either on emission of the spray or as the spray travels to a carrier. In particular, the words “spraying a water jet” or similar terms are not meant to imply that the water jet breaks apart into droplets but merely that a water jet was emitted, which includes the possibility of the water jet impacting a carrier as a substantially continuous stream of water.

### Overview

Some systems and methods for growing algae on stationary carriers were

described in patent application PCT/CA2024/050008, Carriers for Growing Algae, published as International Publication Number WO 2024/152103 A1 on Jul. 25, 2024, which is incorporated herein by reference, and substantially repeated herein.

As described in its summary, which is repeated in the following paragraphs, the specification of the PCT application mentioned above describes a system or device that can be used to grow algae substantially in a biofilm attached to a carrier. In some examples, the carrier is stationary.

The carrier has one or more vertically extending surfaces. Optionally, the vertically extending surface is inclined from the vertical. The surfaces may be planar or curved in one or two dimensions. The average inclination, measured on a straight line between the top and bottom of a surface, may be at a small angle, preferably between 2°-10°, most preferably between 3°-7°, to vertical. The one or more surfaces are vertically extending in the sense that their extension vertically is multiple (i.e. 3-50 or 8-20) times greater than their extension in at least one horizontal direction.

The surface may be configured to occupy a two-dimensional space or three-dimensional space. In the case of one or more surfaces occupying a two-dimensional space, carrier may be in the form of many discrete units, each having one or two surfaces, for example a V or U or an inverted V or U., or the carrier may be in the form of a continuous (or at least long) sheet with many (i.e. 3-1000) surfaces, for example a wave or an accordion. In the case of one or more surfaces occupying a three-dimensional space, the one or more surfaces may define for example a cylinder, cone, prism or pyramid. The surfaces may include a bottom of the three-dimensional space or the bottom may be omitted. The surfaces may include a top of the three-dimensional shape or the top may be omitted. Optionally, an upper part of a three-dimensional space may be truncated, for example an apex-truncated cone or pyramid. Two or three-dimensional spaces may be right (i.e., symmetrical about a vertical axis or one or more vertical planes) or oblique. In preferred examples, the carrier is in the form of an open-bottomed, apex-truncated, cone or pyramid.

An inclination to the vertical can also be expressed as an aspect ratio, which is defined as the ratio of height over a characteristic distance at the bottom of the carrier (for example, width or spacing between subsequent surfaces for an inverted V, base diameter for a cone, or base width for a pyramid). The preferred range of angles to the vertical corresponds to aspect ratios of about 10/1) (3°) and 4/1 (7°).

The carriers of the invention can be deployed on or over a reactor surface that is substantially horizontal to develop a larger surface area for growing algae per unit footprint. The ratio of carrier surface area exposed to light over footprint, called specific surface area, is a useful metric to express the intensification factor of the invention; this parameter is also often called the light dilution factor. As will be described in detail below, in some examples carriers of the invention can be deployed to develop a specific surface area of 10 m²/m² or more, optionally up to 25 m²/m².

The specific surface area is a function of the angle to the vertical (or the aspect ratio) and remains the same for any carrier size with the same aspect ratio. However practical considerations help define a range of preferred sizes. At the low end, the size should not be so small that 1) the specific surface area is lost when the carriers are covered with a thick layer of biofilm (i.e., there is bridging of adjacent surfaces by the biofilm), and 2) a space left between carriers at the base to allow water evacuation significantly reduces packing density. These considerations define a preferred minimum height of 0.1 m. At the high end, the size should not be so large that 1) the structure to maintain the carriers in their vertical position becomes complex and costly (given the weight from algal load and possibly wind forces), and 2) the energy required to irrigate the algae is prohibitive (as water may need to be pumped to the top of the carriers). These considerations define a preferred maximum height of 2 m.

So, the preferred carrier height range is from 0.1 to 5.0 m, 0.1 to 2.0 m, 0.2 to 5.0 m, 0.2 to 2.0 m or 0.2 to 1.5 m.

Optionally, the carrier height corresponds to horizontal distances between crests for (for U, V or inverted V) or summits (for cones) that would allow circulation of operators on their top surfaces with or without light machines on wheels for the purpose of carrier installation, maintenance, irrigation or harvesting of algae. Operators may have to wear special shoes (like snowshoes) and light machines may have fat tires to spread their weight over multiple carriers.

The surface of the carrier is not limited to being flat (e.g. V, inverted V) or curved (e.g. cone) but could be pleated, undulating ore otherwise embossed to create local valleys and peaks and thus further increase the specific surface area. Optionally, the surface of the carrier may be formed of a flexible material, for example a fabric.

The carrier of the invention can be composed of one or more layers. The first layer may be solid but preferably, the 1^{st} layer is woven or nonwoven fabric, permeable to water and capable of filtering out algae at least to some extent, optionally after forming a dynamic filtration layer on the fabric. The first layer may be made of a hydrophilic material to improve water distribution over the carrier surface and delivery of nutrients to the algae. Preferably, a 2^{nd} layer is a mesh or screen that create alveoli where algae can grow protected from shearing by flowing water. Third or more layers may also be a mesh or screen. The layers are preferably directly adjacent to each other. Optionally, the layers are spaced apart from each other, for example by 0.1 to 1.0 mm or 0.5 to 10 mm, to provide a volume for biofilm growth between layers. Spacing between layers may be provided, for example, by undulations in one or more layers, by spacers, by variations in rigidity, tension or compression, or by variations in diameter.

The biofilm is kept wet, for example by irrigation, continuously or intermittently. In some examples, irrigation is 0.5-5 min on and 2-10 minutes off or 5-15 minutes off, for example 1 min on and 9 min off. Preferably, irrigation involves adding water near the top of the carrier and letting the water trickle down the surface. Alternatively, water may be applied, for example by a spray or drip, to some or all the surface of the carrier. Optionally, the biofilm may be kept wet by adding water to the outside of the carrier, for example by adding water directly to an outer surface of the carrier or to an algae film on the outside of the carrier.

The surface of the carrier may contain undulations or ridges whose purpose is to divert and redistribute the irrigation flow and/or to prevent channelling. The carriers can be deployed over a reactor that contains water, but

preferably they are deployed over a gently sloped surface where the irrigation water can be collected and recycled. Carriers can be self-standing, or preferably mounted on a structure where they are hanging. A carrier mounting structure can also include a hydraulic network used to convey irrigation water. Alternatively, the biofilm may be wetted by temporarily submerging the carriers, for example by dipping the carriers into water below the carriers or by temporarily raising the surface of water above the carriers.

In a reactor used to grow algae, the carrier's surface is exposed to light and in contact with a source of gaseous CO₂. The light is preferably natural, and the source of CO₂ is preferably air. The reactor has means to add feed water and remove effluent water. Feed water can be make-up water containing nutrients and effluent water can be water depleted in nutrients. Feed water also can be wastewater and effluent water can be treated wastewater. The reactor has means to recirculate water at a rate that allows keeping the algae wet and fed with nutrients, without abrading the algae from the biofilm.

Algae are periodically harvested from the carriers. For example, algae can be harvested by spraying the biofilm surface to detach the algae. The algae are collected after they detach from the carrier surface.

In some examples, the carriers may offer one or more benefits. First, the carriers may have a large specific surface area. For example, a V, an inverted V or a cone with an aspect ratio of 8 (angle to vertical) 3.6°) has a surface area for growth over footprint of approximately 16/1. This specific surface area represents a potential intensification factor (ratio of algae production per unit footprint relative to that of a raceway pond) as the rate of algae growth is typically proportional to surface area, limited by light or CO₂ absorption from the ambient gas. Second, an angled surface area is easy to access from above for irrigation of the biofilm. Third, an angled surface area, when preferentially combined with a water permeable carrier fabric speeds up biofilm establishment and growth by having a portion of the irrigation water flow through the carrier. An angled surface may also contribute to suspended solids removal when used for wastewater treatment. Fourth, when using natural light, an angled surface might promote better light exposure to an array of carriers as the sun moves through the sky.

The algae carriers may be used to grow microalgae for one or more purposes, including 1) as food for human consumption or for the extraction of nutraceuticals, 2) as feed for aquaculture and animals, 3) for their fertilizer value, 4) for their energy contents to be converted into biogas or liquid fuel, 5) to biologically capture carbon dioxide for the purpose of sequestration, and 6) for wastewater treatment.

The reactor can be used to grow algae for their intrinsic value, optionally using the minimum amount of feed water containing nutrients, or it can be used to treat wastewater optionally while collecting algae as a valuable by-product and/or for carbon sequestration. The reactor design and its modes of operation are similar for both applications.

The shape of the reactor is not critical, but rectangular reactors are preferred. The reactor can be of any size, preferably in the range from 200-10,000 m², most preferably from 500-5000 m². For a large plant, multiple reactors can be used.

Overall, it is desirable that the reactor have minimum permanent supports and/or equipment overhead that could provide shade to the carriers.

The reactor may include a process tank or be connected to a process tank. When growing algae, a fresh nutrient solution is added to the process tank, and an equivalent amount of bleed water is taken out of the process tank, minus evaporation. When treating wastewater, flow through the process tank may be a function of the treatment capacity of the reactor for specific pollutants (e.g., nitrogen, phosphorus, BOD/COD). Water may be pumped from the process pump to an irrigation system continuously or intermittently to provide nutrients to the algae biofilm and to keep the algae biofilm wet.

A spraying harvesting network is also attached to the overhead distribution system.

Ambient or modified air may be used to provide gas transfer into the biofilm, CO₂ for algae growth and optionally O₂ for bacterial respiration (for example when treating wastewater with a mixotrophic biofilm at night). The reactor may define a path for air flow through the carriers which can be by natural convection or forced flow. Spray irrigation can also provide an opportunity for transfer of CO₂ and O₂ from/to the air

Optionally, the reactor can be covered or enclosed, for example by a greenhouse, to improve year-round operation at high latitude. In this case, it may be desirable to minimize the overall height of the reactor. When enclosed, the air inside can be enriched with a source of CO₂ to boost algae production

Artificial lighting can be added to the reactor to support photosynthesis during times of low or no solar irradiation (i.e. night, winter, dawn/dusk).

Further to the summary of PCT/CA2024/050008 included above, microalgae can be grown in a biofilm on a carrier surface. Carriers in the form of cones with surfaces at a small angle to the vertical can deploy large surfaces area per unit footprint, for example more than 10 m²/m². This correspondingly increases the areal production of algae by making better use of natural light which is typically available in excess through the principle known as light dilution.

Carriers can range in height, for example from 0.1 to 5.0 m, 0.2 to 5 m or 0.2 to 2.0 m. Carriers can have a porous and uneven surface to facilitate algae attachment.

The carriers can be deployed over a gently sloped floor where the irrigation or harvesting water after contacting the carriers can be collected. Carriers can be mounted on a structure where they are self-standing or hanging to form a carrier bed.

Microalgae can be grown in repeated cycles of irrigation and harvesting phases. During the irrigation phase, water containing nutrients is sprayed continuously or intermittently in small droplets that do not carry sufficient energy to strip the biofilm. This irrigation phase may last several days, typically between 3 to 12 days, preferably between 5 to 8 days. The duration of the irrigation phase has an impact on algae composition; it can affect the concentration of nutrients and the proportion of proteins, lipids, carbohydrates. At the end of an irrigation phase, microalgae may have grown to represent up to 100 dry g per m² of carrier surface, at a biofilm concentration ranging between 4% to 15% by weight.

It is desirable to harvest the microalgae while limiting their dilution with water. However, mechanical means to harvest algae can be complex and costly. In an example in application PCT/CA2024/050008, algae were harvested hydraulically by hand spraying with a garden hose. While this method allowed recovery and quantification of the algae produced, it is not scalable to a large plant. A hydraulic harvesting method preferably i) minimizes algae breakup into small particles, ii) avoids diluting algae in a large volume of water, iii) provides control over the fraction of the biofilm removal to allow regrowth, and/or iv) avoids damaging the carrier surface.

This specification describes a novel hydraulic method and apparatus to harvest algae. The harvesting method involves stripping the biofilm from a carrier using a jet of water. The water jet may spin, optionally in rapid movement, and translate. An apparatus may include a manifold holding one or more rotating nozzles and a bridge holding the manifold that translates the nozzles across a carrier bed.

In some embodiments, a method for dislodging algae from a plurality of carriers includes spraying a water jet at the carrier. The water jet is sprayed from one or more nozzles and travels through air to reach the carrier. The one or more nozzles rotate about an axis oblique to the water jet or translate in a direction oblique to the water jet or both. Where the water jet rotates and translates, an axis of rotation may be oblique, optionally perpendicular, to the direction of translation. The water jet may strip parts of an algae biofilm from the carrier. The water jet may rotate in a rapid movement, optionally completing many rotations for every translation. The water jet may be created from a nozzle, optionally a nozzle that spins in reaction to the water jet.

The water jet is preferably a substantially solid (i.e. continuous) stream. The breakup of the jet into droplets is undesirable as it greatly diminishes its stripping efficiency. Generating and maintaining a solid stream also ensures that the jet remains efficient with the tallest carriers of the invention.

The impact force of a water jet can be controlled by changing flow rate and velocity. The jet should have an impact force sufficient to strip the biofilm while minimizing the dispersion of algae. Algae are typically held together cohesively in the biofilm, and it is desirable that they come off as large agglomerates or blobs. Obviously, the impact force should also be such to avoid damaging the carriers.

Optionally, one or more water jets move over the surface of the carrier bed, optionally in a continuous and/or rapid movement that allows the water jets to reach substantially the entire surface of the carrier bed. These water jets strip the algae from substantially all the surface of the carriers.

The rapid movement of the water jet can be achieved with a combination of two motions: spinning of the nozzle itself and a horizontal translation movement of the nozzle or a nozzle assembly over the carrier bed.

It is preferable to harvest only a fraction of the algae in order to leave some to start a new growth cycle. The target recovery during each harvest event is between 80-95%. Partial harvesting is facilitated by the nature of the carrier surface, which is designed to retain some algae, and by controlling the flow per nozzle, the nozzle spinning and the translation movements.

It is desirable to minimize the duration of the harvest and the amount of water used to avoid diluting the harvested algae.

Algae stripped from the carriers are collected on the reactor floor and directed hydraulically to a collection tank where they can be further concentrated (for example, by settling) and processed. Alternatively, algae may be scraped, pushed, blown or vacuumed from the reactor floor.

### Carriers

The description below uses examples of carriers that are either an inverted V or a cone. In these examples, the surface is inclined to the vertical and symmetric around a vertical plane or a vertical axis. Carriers in other two-dimensional or preferably three-dimension shapes, having other forms vertically extending surfaces, may also be used.

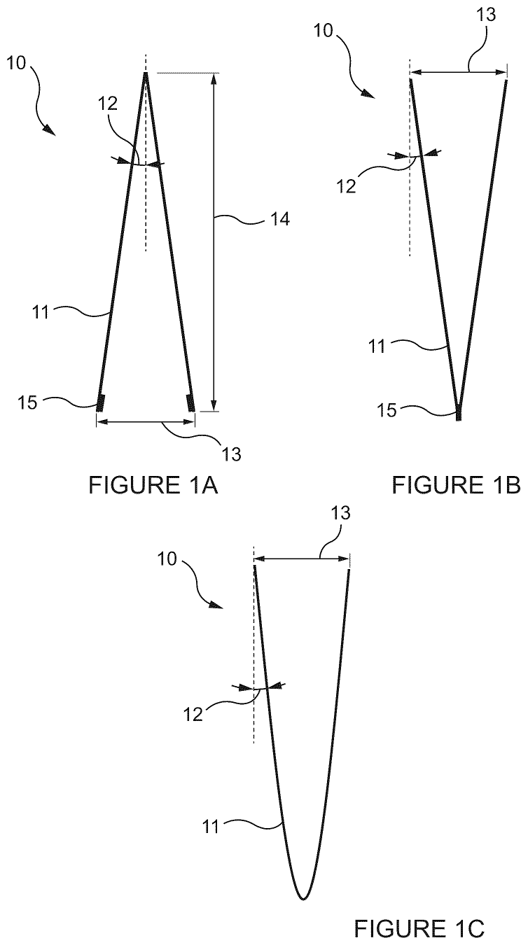

FIG. 1 represents 3 examples of the cross-section of the general configuration of a stationary carrier 10. FIG. 1a) is an example of an inverted V or a cone or pyramid; FIG. 1b is an example of a V shape or inverted cone or pyramid; and FIG. 1c is an example of a parabolic U-shaped carrier or an inverted cone or pyramid, all with the same preferred angle to the vertical and aspect ratio.

Angle 12 is defined as the inverse tangent of ratio of half of the width 13 divided by the height 14, independent of whether the actual carrier surface is a straight line or not.

Angle 12 is preferably between 2°-10°, most preferably between 3°-7°. Angle 12 defines an aspect ratio which is equal to height 14 divided by width or diameter 13. The preferred range of angles from the vertical corresponds to aspect ratios preferably between about 14:1 and 3:1, most preferably between 10:1 and 4:1. Carrier 10 can be self-standing if its surface has a solid layer capable of maintaining its shape or if its surface is mounted on a frame. For this case the carrier could be sitting on the reactor surface, supported from below. Preferably, carrier 10 is suspended from its top portion, supported by one or several posts (not shown). Alternatively, carrier 10 could be hung from a cable or a structure above, as long as that structure does not create significant shade on the carrier surface.

Carriers supported from their top portion can be made from a flexible material if, for example, they have a relatively rigid or heavy structure at bottom 15 to maintain their shape. Carriers designed this way could be self-levelling. In the example of FIG. 1c, the U-shaped carrier is supported from its top portion and the flexible fabric is loosely hanging; its actual shape can be a function of its weight which may change with biofilm thickness.

As it relates to carriers symmetrical to a vertical plane, it will be understood that the examples in FIG. 1 represent a single fold of a continuous carrier that would be similar to an accordion or a pleated fabric.

Carrier 10 can have a section of its top portion cut-off or truncated, typically 5-20%, to create a space sufficient to contain a support mechanism and potentially a water distribution means for irrigation.

The nature of the carrier surface is not critical as it is well known that algae can attach to almost any surface given enough time. However, it is preferred that the carrier has one layer made of a fabric with the following characteristics: non-biodegradable, strong, UV resistant, porous, and hydrophilic. Strength is required to resist the algal load and any applied forces (e.g., wind, water sprays). UV resistance is preferable for durability. Porosity and hydrophilicity are preferable to allow water passage during start-up to accelerate biofilm establishment and to transport nutrients. Porosity should preferentially be in a range between 25-75%, with pore sizes 2-100 μm or 2-20 μm. Hydrophilicity can be measured by water absorption capacity, which may be at least 20g/100 g of material, as measured for example by method ISO2417. Preferred materials for the first layer include woven or nonwoven fabrics from material including, but not limited to polyester, polyethylene, polypropylene, nylon, etc. The material for the first layer could also be chosen among low carbon footprint materials such as cotton, hemp, jute, linen/flax, sisal, bamboo, coir, grasses, etc. The material for the first layer may be rigid or flexible.

An optional second layer could consist of a mesh or netting material

capable of creating alveoli to retain algae in the biofilm and prevent premature slough off. Preferred materials for the second layer may be similar to the first layer but with grid openings of 1-10 mm. Third or more layers may be similar to the second layer.

An alternative to a second layer consists of embossing, pleating or otherwise shaping the first layer to create the desirable alveoli.

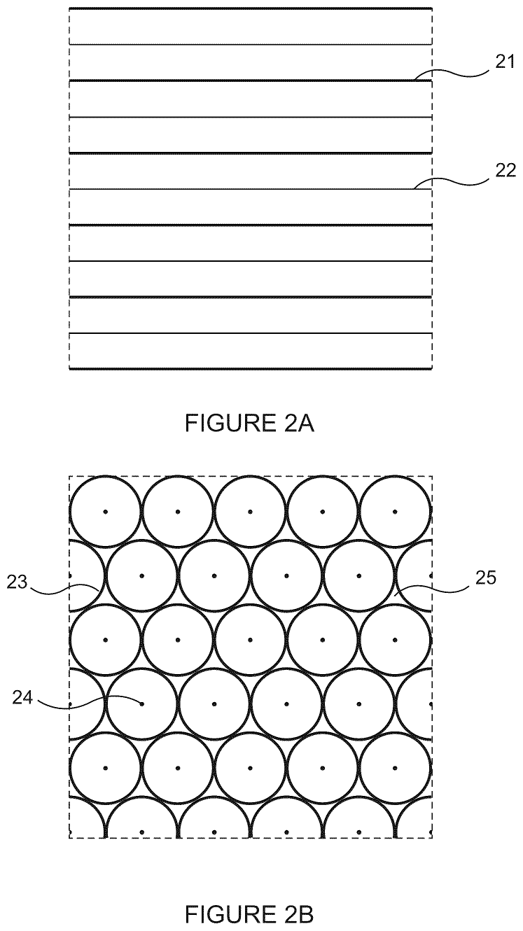

FIG. 2 shows how inverted Vs and cones can be deployed to cover 1 m² of the surface of a reactor. These were drawn to scale to represent carriers with an aspect ratio of 8 (angle from vertical of 3.6°)having a height of 160 cm and a base of 20 cm. In FIG. 2a, the bases of inverted Vs are represented by thick lines 21 and the crests by thin lines 22. There are 5 carriers per m² of reactor surface area, each measuring 1 m long. This accordion pattern has a surface area for biofilm growth of 16 m², or a specific surface area of 16 m²/m². FIG. 2b shows cones in hexagonal packing (rows offset by half a diameter). There are 28.9 cones per m² with a specific surface area of 15.4 m²/m².

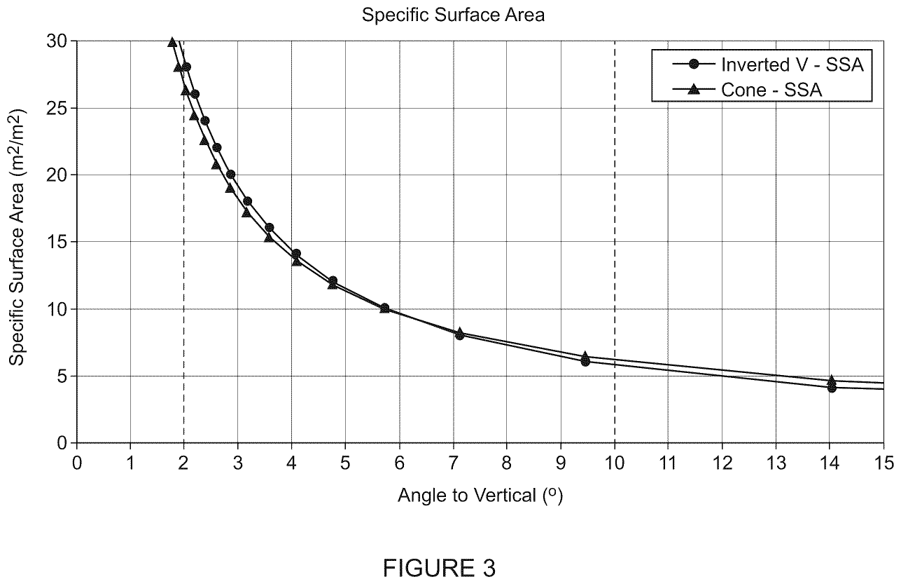

FIG. 3 is a graph showing the theoretical maximum specific surface area as a function of the angle to vertical for inverted Vs and cones packed as illustrated in FIG. 2. The range of preferred angles (2°-10°) is within the vertical dotted lines while the most preferred angles (3°-7°) is within the vertical solid lines. This graph shows that the inverted V has a slightly higher theoretical maximum specific surface area as compared to the cone within the most preferred range.

The actual specific surface area deployed in a reactor is typically lower than the theoretical maximum surface area represented by FIGS. 2 and 3. For example, a portion at the top of the inverted V and the cone may have to be sectioned or truncated to position supporting brackets and/or irrigation means. At the bottom, a gap may be needed between inverted Vs for flow of irrigation and harvesting water to the reactor floor below. For cones, hexagonal packing may leave a sufficient flow gap, for example when the cones are within a size range of 0.1 to 5.0 m or 0.1-2 m, but spacing can be increased to achieve any gap size.

The table below is an example of theoretical maximum and actual specific surface areas for carriers within the most preferred range, with a height of 160 cm and base of 20 cm. A 15% section is cut off at the top for both the inverted V and the cone, leaving a space of 3 cm for attachment to a supporting structure and/or irrigation means. At the bottom, there is a gap of 2 cm between inverted Vs, but there is no need for an additional gap between rows of cones as there already are openings of about 3 cm between cones as illustrated in FIG. 2b. It should be noted that the carrier projected surface area of 0.907 represents the maximum packing density for circles in a hexagonal pattern. The actual specific surface area is reduced to 12.4 and 15.1 m^{2/}m², respectively for the inverted Vs and the cones. This analysis shows that, for the same aspect ratio, the cone offers a 22% higher actual specific surface area.

The actual specific surface area can be used to extrapolate the surface production of a carrier to an aerial production.

| | Units | Inverted V | Cone |
|---|---|---|---|
| Aspect ratio | | 8 | 8 |
| Height | cm | 160 | 160 |
| Base | cm | 20 | 20 |
| Theoretical Maximum Specific | m²/m² | 16.0 | 15.4 |
| Surface Area | | | |
| Portion cut-off at the top | | 15% | 15% |
| Dimension where cut-off | cm | 3.0 | 3.0 |
| Net height | cm | 136 | 136 |
| Bottom gap | cm | 2.0 | 0.0 |
| Carrier project surface | fraction | 0.907 | 0.907 |
| area coverage | | | |
| Actual Specific Surface Area | m²/m² | 12.4 | 15.1 |

### Reactors

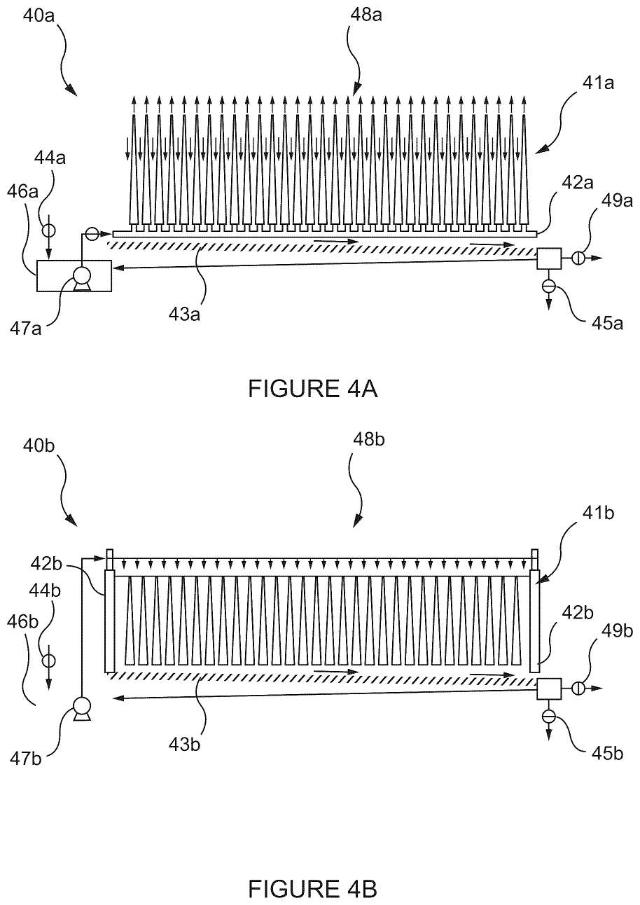

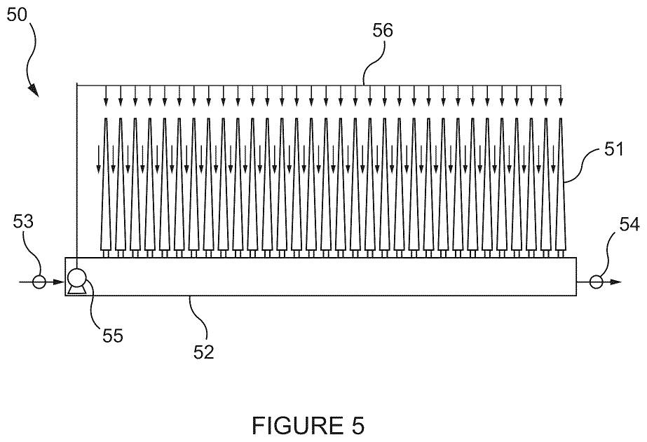

FIG. 4a is an example of a reactor 40a where the carriers 41a are mounted on a hydraulic network 42a. The hydraulic network is substantially leveled but is mounted on a reactor floor 43a which is sloped to recover irrigation water. Reactor 40a has means to add feed or make-up water 44a containing nutrients and to withdraw effluent 45a depleted in nutrients. Feed 44a is added to a tank 46a which contains irrigation water, from which a pump 47a can distribute it to the carriers through hydraulic network 42a. Each carrier 41a is mounted on a standpipe having a distribution cap 48a which allows distribution of irrigation water flowing upwards from hydraulic network 42a through the standpipes and the distribution caps 48a to the outside of the carriers 41a near the top of the carriers 41a. From there, irrigation water trickles down the outside of the carriers and is collected on sloped floor 43a from where it flows back to tank 46a.

FIG. 4b is an example of a reactor 40b where carriers 41a are suspended from cables 41b or other suspending structures such as beams, which may be arranged in parallel or in a grid. The cables 41b are attached to support structures 42b located on opposite sides of the reactor 40b. The carriers 41a are self orienting (i.e. they hang generally vertically) and are mounted at substantially the same elevation (with some variation for the cable sag). The reactor floor 43b is sloped to recover irrigation water. Irrigation water is distributed to the top of the carriers from above through an irrigation system, in the example shown having nozzles mounted on a moving bridge 48b. In this embodiment, bridge 48b travels on top of support structure 42b, but alternative mounting arrangements are possible. In the example shown, the bridge 48b moves at right angle to the direction of the suspension cables (i.e., in and out of the page). As the moving bridge 48b travels across the bed of the reactor 40b, individual carriers 41a are sprayed with water intermittently but the water is dispersed over the bed area (i.e. floor 43b) of the reactor 40b. Other hydraulic functions of reactor 40b are like reactor 40a and will not be repeated here for brevity.

In the embodiments represented by FIG. 4a and FIG. 4b, inorganic carbon required for algae growth is mostly transferred directly to the biofilm from ambient gas, but it could also be dissolved into tank 46a,b. The reactor 40a,b may behave like a completely stirred tank reactor (CSTR) if the recirculation of irrigation water is sufficiently greater than the feed/effluent flow. In some cases, especially involving wastewater treatment, it may be desirable to deploy multiple reactors like reactor 40a,b in series to approach a plug flow mode of treatment.

In the mode of operation described above, algae slough off from the carriers when the biofilm becomes too thick, and they are recovered with the effluent.

As an alternative, reactor 40a, b can be turned into harvesting mode by stopping feed 44a,b and diverting effluent 45a,b to a harvested algae stream 49a,b. In this mode, the carriers may be sprayed to remove algae from the surface of the carriers and to recover algae in a concentrated form. The carriers 41a and algae films attached to the carriers 41a are wetted by irrigation water applied to the outside of the carriers 41a. In these examples, irrigation water does not flow outwards through the algae supporting surfaces of the carriers 41a to wet the algae films.

FIG. 5 is an example of a reactor 50 where the carriers 51 are mounted on top of a tank 52. They can be mounted on posts (as shown) or suspended from a suspending structure (similar to FIG. 4b, not shown). Reactor 50 has means to add feed or make-up water 53 containing nutrients and to withdraw effluent 54 depleted in nutrients. Feed 53 is added to tank 52 which contains irrigation water, from which a pump 55 can distribute it to the carriers through a fixed hydraulic network 56 with nozzles directed at the tops of carriers 51 (as shown) or with fixed wider angle spray heads to disperse water across the bed area of the reactor 50, or through a moving bridge 48b similar to FIG. 4b (not shown). Irrigation water trickles down the carriers and flows back to tank 52. One or more features of reactor 50 may be substituted or combined with one or more features of reactor 40a or reactor 40b.

Alternatively, carriers may be irrigated by temporarily immersing the carriers in water. For example, in a modified reactor 40b, the carriers 41b may be suspended over a tank 52 as in FIG. 5 (but preferably a deeper tank) in place of the reactor floor 43b. One end of the cables 41b may be attached to a bar that is pulled tight or relaxed by a hydraulic cylinder attached to one of the support structures 42b. When tension in the cables 41b is released, the carriers 41a are lowered into the water in the tank 52. Tightening the cables 41b lifts the carriers 41a back out of the water. The action of dipping the carriers 41a may release at least some of the algae from the carriers 41a but additionally or alternatively algae may be harvested from the carriers 41a by spraying water at the carriers 41a. The carriers 41b may also be intermittently lowered into water for other purposes, for example to protect the carriers 41b or algae from inclement weather if the carriers 41b are deployed outdoors. In another option, a tank 52 may have sides extending above carriers 41a or 51. The water level in the tank 52 may be raised and lowered to intermittently immerse the carriers 41a or 51.

### Experimental Methods

Validation experiments were done with a mixed culture of Chlorella vulgaris obtained from the Canadian Phycology Culture Centre (Waterloo, Ontario) and Tribonema minus obtained from Cal Poly (San Luis Obispo, California). Micro photos of the algae were taken with a AmScope M170C-E 40X-1000X Dual LED Portable Compound Microscope with Camera.

The algae were grown using Miracle GRO 24-8-16 at a concentration of 200 mg/L as a source of nutrients and, soda (NaHCO₃) at a concentration of 200 mg/L as an additional source of inorganic carbon and to increase pH and promote better uptake of atmospheric carbon dioxide. These chemicals were dissolved in de-chlorinated tap water from the City of Hamilton, Ontario.

Experiments were conducted in different reactors.

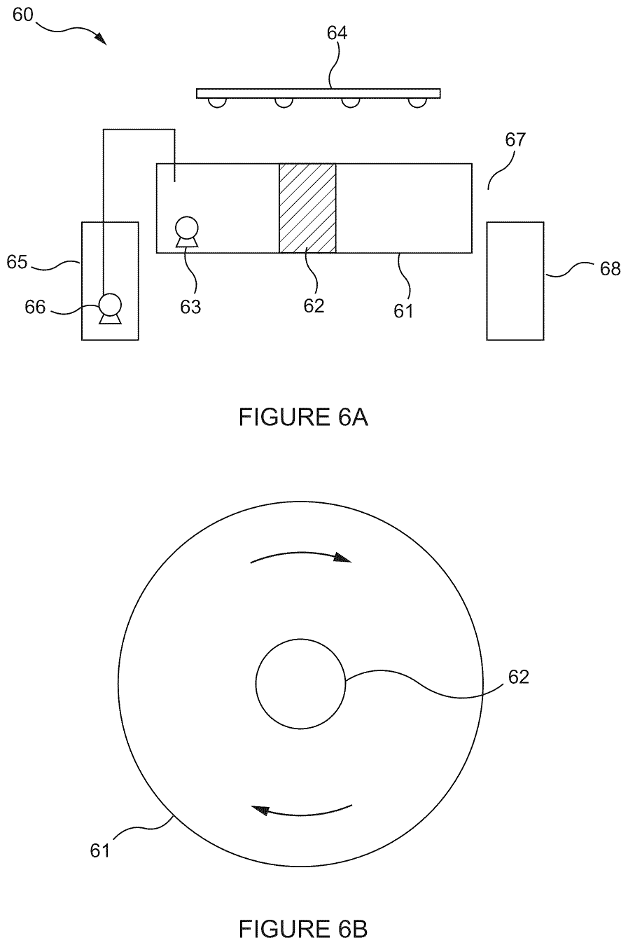

The reactor shown in FIG. 6 was dedicated to growing algae in suspension as a control. FIG. 6a shows a cross section; FIG. 6b is a plan view of the tank and centre isle showing the flow plan. Reactor 60 consisted of a 50 L tank 61 with a center isle 62 and a net surface area of 0.47 m². A submerged pump 63 was used to create a circulation movement in the tank. The pump, Coral Box model QPS9, created a velocity of 20-30 cm/s, sufficient to prevent settling of suspended algae, and could also be used in a pulsing mode. Eight LED lights 64 from FEIT model GLPFS/19W were positioned above the tank to generate a photon intensity of 150 μmol/m²/s on a continuous basis (measured using a Light Scout 3415A meter). A system was put in place to pump a feed solution into the reactor on a semi-continuous basis. It consisted of a feed pail 65, a feed pump 66, an overflow tube 67 and an effluent pail 68. The programmable pump 66 from Moisten Land, model 201, was turned on regularly to generate a flow of 5-10 L/d, which corresponded to a hydraulic retention time of 5-10 days.

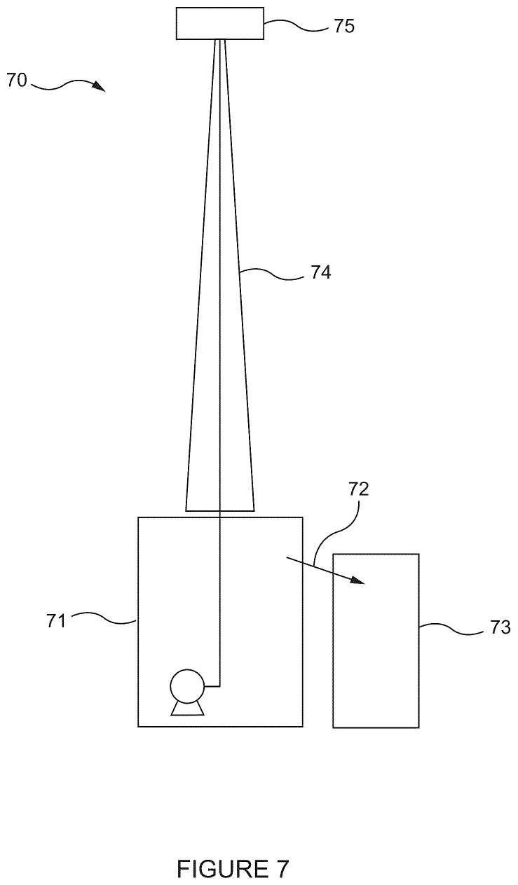

The reactor shown in FIG. 7 was used to grow attached algae with a conical carrier. The reactor consisted of a pail 71 with a working volume of 10L and an overflow tube 72 into an effluent pail 73. A carrier in the form of cone 74 was positioned over the reactor, hanging from a bracket above the cone 74. Feed was added manually to the reactor, 2L in the morning and 2L at night. An irrigation water distribution box 75 was located above the cone 74 and had 16 holes of 1.6 mm diameter each equally spaced around its perimeter (on a 4 cm diameter). The holes of the irrigation water distribution box 75 were located outside of the perimeter of an adjacent part of the cone 74 such that irrigation water was delivered to the outside of the top of cone 74 and then trickled down the outside of the remainder of the cone 74. An aquarium pump (Cadrim rated 1200 L/h) submerged in pail 71 pumped irrigation water through a tube connected to distribution box 75. The instantaneous flow rate was set with a restriction valve to 30 L/h and was used intermittently, 1 min on, 5 min off. This generated a trickling flow along the surface of the cone. Eight LED lights (not shown) identical to those used for the suspended growth reactor were positioned around the cone to provide light at a photon intensity of 150 μmol/m²/s on a continuous basis.

The carrier was inoculated by filling the reactor with a suspension of algae taken from the effluent pail of the suspended growth reactor, diluted 1/1 with tap water. The pump was then started in recirculation loop for half a day before starting to add feed.

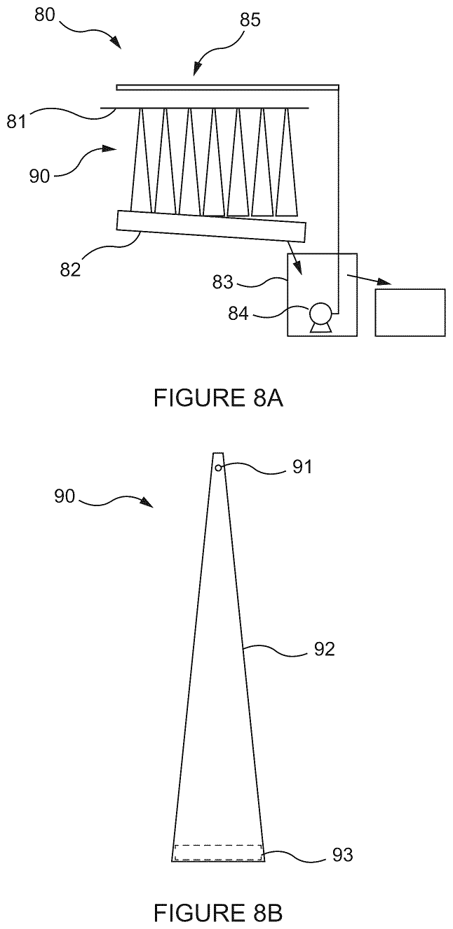

Reactor 80 shown in FIG. 8a was used to grow algae on carriers in the form of fabric cones 90 suspended on cable 81. The reactor consisted of tray 82 set with a slope and an opening to collect irrigation water and return it to pail 83. Pump 84 was used to recirculate water from pail 83 to irrigate the cones 90 through 6 stationary nozzles 85 located above the cones. Pump 84 was a small misting pump capable of generating a flow of 2.5 liters per hour through misting nozzles 85. The pump was controlled with a timer and was on for 30 seconds and off for 2 minutes. Pail 83 contained 10 liters and was fed with 2 liters per day of a fresh solution of nutrients as described above. It overflowed into effluent pail 86. Four LED lights 87 identical to those used for the suspended growth reactor were positioned on both sides of the cones 90 to provide light at a photon intensity of 100 μmol/m²/s on a continuous basis.

FIG. 8b shows further details of the cones 90. The surface of the cone is formed by flexble fabric 92. Each cone 90 had a fixture 91 for attaching the cone 90 to the cable 81. In the example shown, the fixture 91 is a grommet inserted through both sides of the fabric 92. In use, the cable 81 passes through the fixture 91. The bottom of the fabric 92 is kept open by a rigid frame 93. In the example shown, the frame 93 creates a circular opening. Cone 90 is described further in the experimental section below.

Water in the reactors was monitored daily for pH and temperature using a Hanna 98129 tester, and turbidity using a Hanna HI 3415A meter.

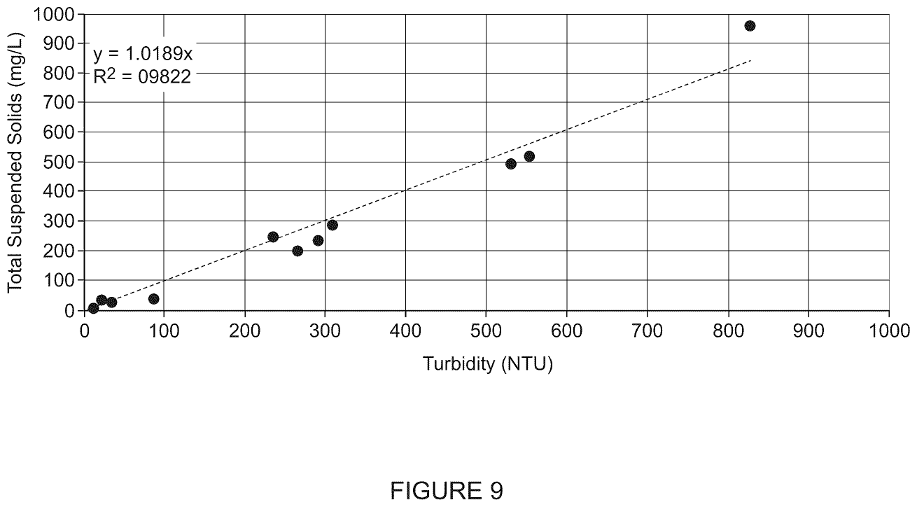

Turbidity was converted to suspended solids using the correlation shown in FIG. 9. Total suspended solids (TSS) were measured using a standard method by ActLabs Agriculture, Ancaster, Ontario. The correlation shows a roughly 1 to 1 relationship between turbidity and TSS, i.e., 1 NTU=1 mg/L. It was assumed that a TSS measurement represent the dry mass of algae without any other correction.

### Experimental Carriers

Three cones of identical dimension were built and tested sequentially in the reactor shown in FIG. 7. They had an aspect ratio of 8 (angle to vertical of 3.6°with a diameter at the base of 18 cm and an 18% section cut off at the top, which left a net height of 118 cm. They had a biofilm growth surface area of 0.42 m², a footprint of 0.028 m², corresponding to a specific surface area of 15.0 m²/m². The cones could be attached to the irrigation distribution box described above (75 in FIG. 7) and thus hang over reactor 70.

Cone 2511 was built with 3 layers. The first layer was a solid sheet of polystyrene, 0.76 mm thick, which was glued in the shape of a cone. This first impermeable layer was covered with a second layer of self-adhesive fiberglass tape from a local hardware store. The third layer was a material called Synlap, a synthetic burlap with a large mesh used to cover shrubs over the winter.

Cone 0212 did not have a solid base but was built with 3 layers of fabric obtained from a local store. The first layer was a tight weave of polyester fabric. The second layer was polyester tulle with openings of about 1 mm. The third layer was polyester poly-mesh with openings of about 5 mm. These fabrics were cut to a pattern, sewed together with stitch lines about 30 mm apart and then sewn into a cone. At the top, fabric was glued to a short piece of pipe for attachment to the reactor. At the bottom, belt loops were sewed on the inside to insert a plastic ring of 5 cm wide to expand the base into a circle. As a result of the stitch lines and differences in the material of the layers, wrapping the layers in a cone caused the layers to separate from each other to a varying degree, in some places by a few mm.

Cone 1012 was identical to Cone 0212 with the exception that the stitch lines were about 10 mm apart to limit separation of the layers of fabric from each other. A plastic ring was also added at mid-height to help redistribute irrigation water along the circumference of the cone and reduce channeling.

The hydrophilicity of the first and third layers of fabric used to make cones 0212 and 1012 was measured by the method described above. Both fabrics spontaneously absorbed water and had an absorption capacity of about 150 grams water per 100 grams of fabric.

Smaller cones were tested in the reactor described in FIG. 8a. Cone 1108 shown as cone 90 in FIG. 8b had an aspect ratio of 8, with a truncated length of 36 cm and a diameter at the base of 6.4 cm. It was sewed together from 2 layers of fabric, a woven polyester as base and an open mesh tulle as second layer. Seven (7) cones 1108 were hung side by side on a cable to provide total surface area of 0.35 m², a footprint surface area of 0.028 m2, corresponding to a specific surface area of 12.4 m²/m².

### Experimental Result-Algae

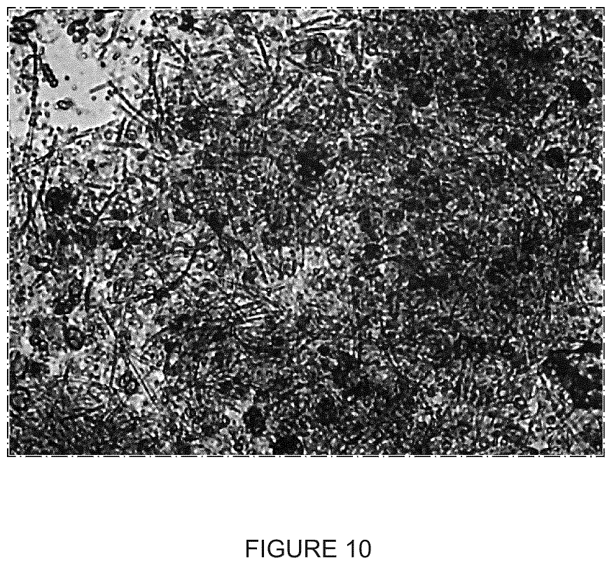

FIG. 10 is a microscope picture of algae from a biofilm showing Chlorella vulgaris (round shape) and Tribonema minus (filamentous shape). There is no visible bacterial growth or contamination such as would be present in wastewater.

Experimental result-Algae grown in suspension

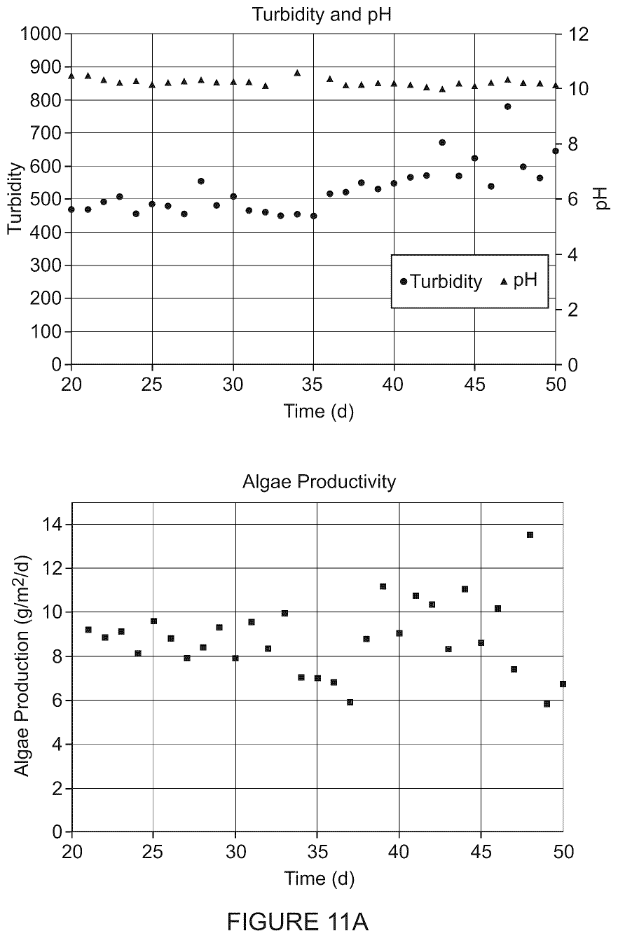

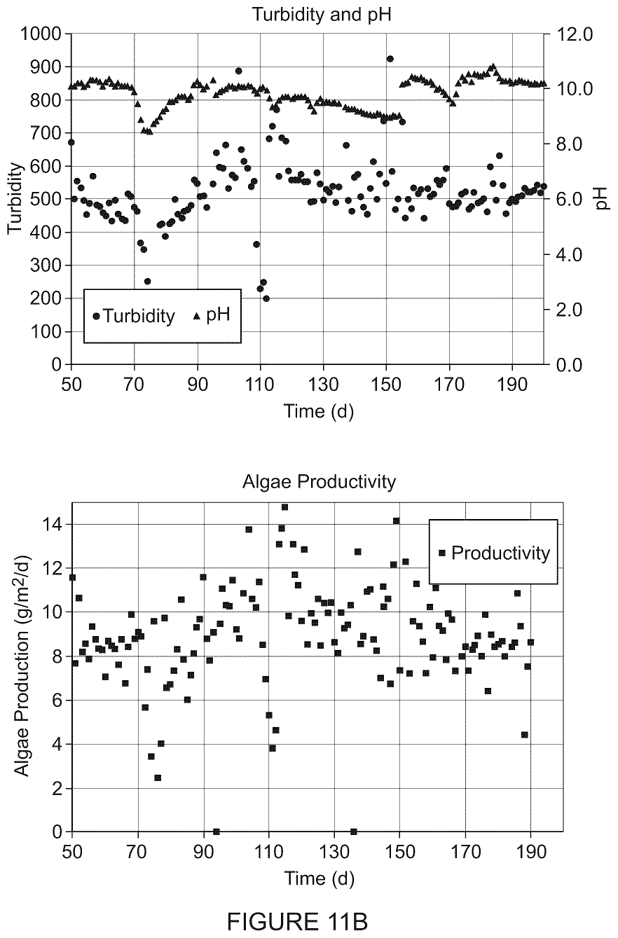

FIG. 11a shows the results of algae growing in suspension over a period of 30 days corresponding to the period when cones 2511, 0212 and 1012 were tested. pH was stable, just above 10.0 for the duration of the experiment. A high pH is an indication of a healthy algae population as the inorganic carbon in solution (from alkalinity and added sodium bicarbonate) has been mostly depleted. Turbidity ranged between 500-600 NTU. The temperature was stable, averaging 20.5° C. over the duration of the experiment. Turbidity was converted into algae production rate using the correlation presented in FIG. 9. Algae production averaged 8.6 g/d/m².

FIG. 11b shows the results of algae growing in suspension over a period of 150 days corresponding to the period when cones 1108 were tested. pH was more variable due to reactor upsets, but generally around 10.0 for the duration of the experiment. Turbidity and temperature were stable and similar to the previous experiment. Algae production averaged 9.0 g/d/m².

### Experimental Result—Algae Grown on Carriers

Each of the three cones 2511, 0212 and 1012 were tested for a period of 7-10 days. At the conclusion of an experiment, cones were sprayed using tap water and a high-pressure nozzle set to a flow rate of 1L/min to remove the algae biofilm. The biofilm was collected in a suspension with the wash (sprayed) water. The algae suspension was blended to break up large particles and diluted to estimate the mass of algae from turbidity measurement.

The primary purpose was to determine the algal load (mass of algae per carrier) through a mass balance rather than simulating a harvesting operation. In a harvesting operation, it would be desirable to leave some algae on the carrier to speed up re-establishment of the biofilm when put back into operation.

In order to better estimate the mass of algae that grew on the surface of the carriers, it is necessary to subtract the mass used for inoculation. As mentioned above, the cones were inoculated by filling the reactor (10L) with an algae suspension.

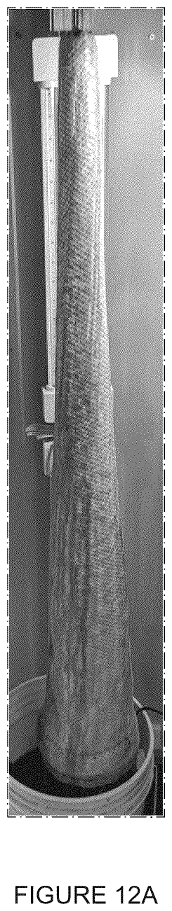

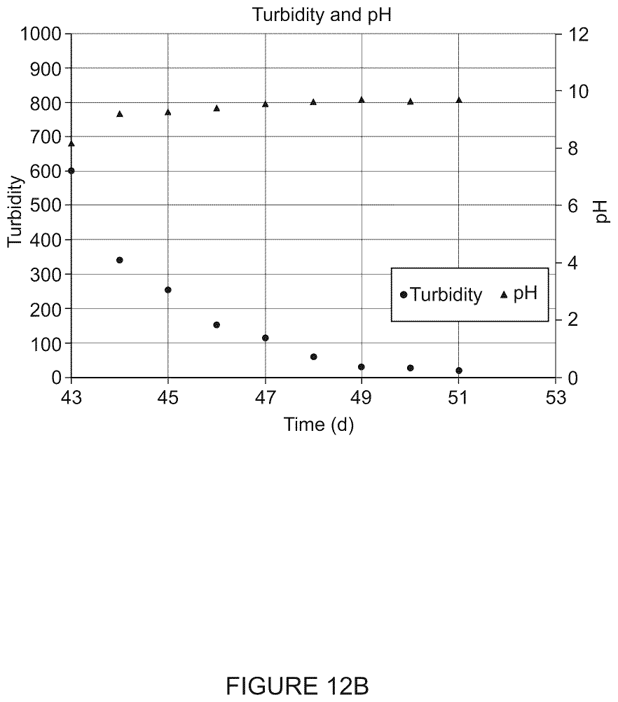

For example, FIG. 12a is a photo of cone 1012 taken 2 hours after inoculation that shows significant algae deposition on the fabric surface of the carrier (though not easily visible in a black and white picture). FIG. 12b is a graph showing that the initial turbidity of 600 NTU quickly dropped, confirming the visual observation of FIG. 12a. Turbidity continued to drop over the duration of the experiment, partly due to deposition, partly due to wash out with addition of fresh feed water (4 L/d). It was estimated that about 3 g of algae were deposited on the cone due to inoculation (half of the mass contained in the inoculation volume of 10L @ 600 NTU). Similar estimations were done for cones 2511 and 0212.

Experimental results are summarized in the table below. In all three

experiments, the algae were recovered at a relatively high concentration of about 5 g/L in wash water. This is about an order of magnitude higher than the concentration in the suspended growth reactor.

The experiment with cone 2511 was stopped after 7 days as significant channeling of irrigation water was observed; surface coverage was estimated visually to be about 40%. The net algae load of 8.1 g was equivalent to a surface production of 2.7 g/m²/d and an aerial production of 41 g/m²/d.

An increase in visual coverage to about 60% was observed with cone 0212. The increased coverage might be a result of eliminating the solid layer or using layers of more hydrophilic fabrics. Surface production increased to 4.4 g/m²/d and aerial production of 66 g/m²/d.

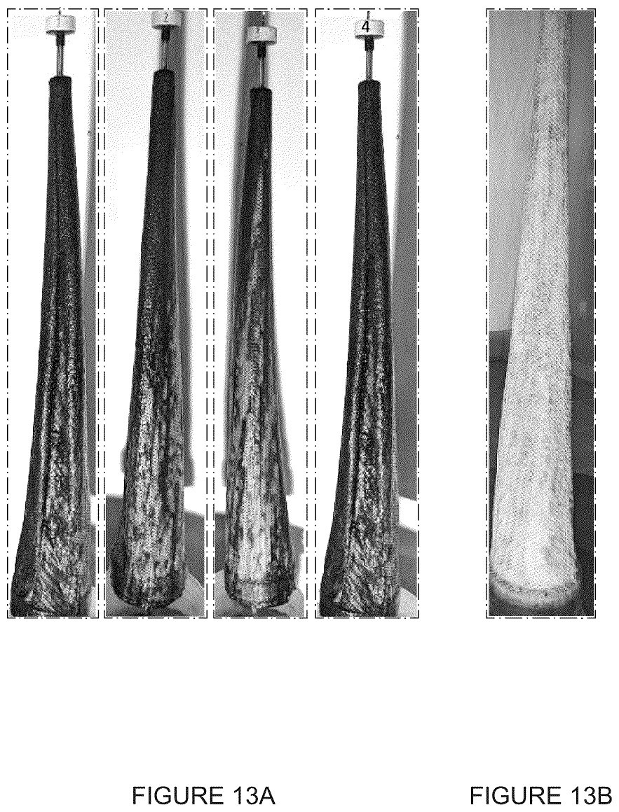

A further improvement was observed with cone 1012, with visual algae coverage increasing to about 80%. The increased coverage might be a result of stitching fabric layers closer together or redistribution of the irrigation water by the half-height ring. Cone 1012 is shown before and after algae harvesting by spraying in FIGS. 13a and 13b. FIG. 13a shows the cone before spraying from 4 different angles; FIG. 13b shows the cone after spraying. Surface production was 7.9 g/m²/d and aerial production of 119 g/m²/d. Surface production is almost the same as the control reactor (8.6 g/m²/d) and aerial production is 14 times larger than the control reactor, which validates the specific surface area concept introduced above as an intensification and scale-up factor.

These experiments involved single carriers exposed to uniform light intensity and did not simulate shade effects that would occur in a packed cone reactor. However, the light intensity of 150 μmol/m²/s would be representative of diluted light reaching the biofilm in a tightly packed cone reactor on a sunny day. While it is impossible to directly extrapolate our results to actual field conditions, the benefits relative to the control suspended growth reactor should hold true under most field conditions.

As indicated in the table below, the cones had an aerial production of over 40 g/m²/d. In comparison, the aerial production of the suspended growth reactor described above was 8.6 g/m²/d.

| Cone | 2511 | 0212 | 1012 |
|---|---|---|---|
| Experiment duration (d) | 7 | 10 | 8 |
| Visual coverage | 40% | 60% | 80% |
| Algae concentration in | 4.7 | 5.0 | 4.6 |
| wash water (g/L) | | | |
| Total algal load (g) | 9.1 | 20.4 | 29.6 |
| Estimated load from | 1.0 | 2.0 | 3.0 |
| inoculation (g) | | | |
| Net algal load (g) | 8.1 | 18.4 | 26.6 |
| Surface production | 2.7 | 4.4 | 7.9 |
| (g/m²/d) | | | |
| Areal production | 41 | 66 | 119 |
| (g/m²/d) | | | |

The turbidity and pH curves of FIG. 12b are worth additional analysis. The initial pH of the inoculation solution, 8.2, gradually increased to above 10, which an indication of a healthy algae population that consumed most of the inorganic carbon in solution. The initial turbidity of the suspension, 600 NTU, gradually decreased to almost zero; this is an indication that suspended solids were removed through contact with the carrier and that algae were not abraded from the biofilm through irrigation. The effluent might not require further treatment before discharge.

Cones 1108 were tested over a period of over 100 days. In this

experiment, the reactor went through repeated cycles of growth and harvest without re-inoculation. Harvesting was done as described above for single cones.

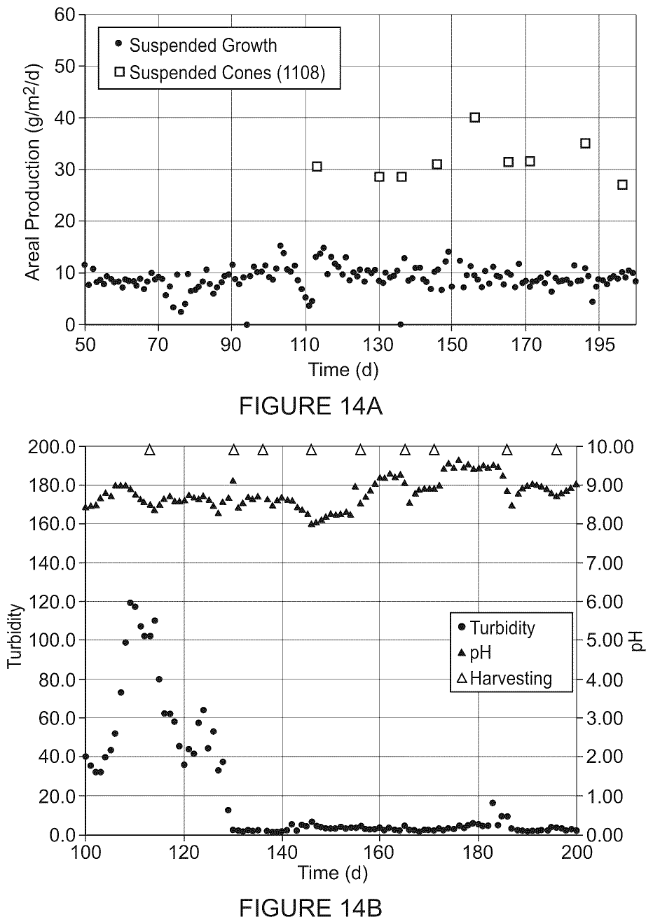

Results are shown in FIGS. 14a and 14b. FIG. 14a shows the areal production in comparison to the suspended growth reactor results. Over the observation period, cones 1108 were harvested 9 times at an average 10-day interval. The areal production averaged 31.7 g/m²/d or about 4 times higher than the production of the suspended growth reactor (9.0 g/m2/d). Effluent water quality is presented in FIG. 14b. Turbidity was high during a few weeks when the cones were sprayed daily with a suspension of algae (from the suspended growth reactor) for inoculation. However, after that initial period, turbidity dropped to below 10 NTU and was not affected by the harvesting events. pH ranged from 8-10; it tended to drop after a harvesting event, but quickly recovered within a few days.

When harvesting, the flow of spray water containing algae was collected into a separate pail (i.e., it was not allowed to flow into pail 83 shown in FIG. 8). The amount of water for harvesting was 8.1 L/m² of cones and the algae concentration was 2.3 g/L, on average. Prior to blending to measure turbidity and suspended solids, the harvested algae suspension was subjected to settling tests and compared to settling of an algae suspension removed from the suspended growth reactor. For the suspended growth reactor, no significant settling was observed over a period of 1 hour. This is attributed to the fact that algae grow as individual cells or small aggregates in suspension and are more or less buoyant. This is a well know characteristic of algae grown in raceway ponds. For the biofilm reactor, the algae quickly settled to about 10% of their original volume, leaving a relatively clear suspension above. This is attributed to the fact that algae grown in a biofilm detach as large blobs that easily settle. The settled algae concentration was about 23 g/L. The volume of water used for harvesting represented about 1.5% of the water treated through the reactor.

### Harvesting

Systems and methods useful to recover algae grown in a biofilm on a stationary carrier are described below. The description and examples below are given, in a non-limiting fashion, using carriers described in patent application PCT/CA2024/050008.

According to some examples in application PCT/CA2024/050008, stationary carriers are deployed as surfaces inclined between 3°-7° to vertical, in the form of inverted Vs or cones that can reach a height of 2m. While these shapes offer large specific surface area (10-25 m^{2/}m²), they remain fully open at the top for light penetration and for performing irrigation and harvesting operations. There are also openings between carriers at the bottom to collect irrigation water and harvested algae.

Most microalgae can attach to a surface and grow in a biofilm. Depending on the application, a biofilm can contain a mixed population of algae of different shapes as well as bacteria (if organic carbon is present), held together cohesively by extracellular polymeric substances.

Stripping (i.e. dislodging) a biofilm from a surface with a water jet requires reaching a minimum impact force. It was discovered that a solid jet where dispersion and the formation of droplets is minimized could maintain its stripping efficiency for several meters from a nozzle head.

The impact force of a water jet is proportional to mass flow times the water jet velocity. Impact pressure is the impact force per unit area of the surface impacted by the water jet. Suitable water jets are formed through highly polished orifices of 1-2 mm at pressures of 2-15 bar, preferably 4-10 bar, generating flow rates of 1-10 L/min. For example, pushing water at a pressure of 4.8 bar through a 1.57 mm orifice generates a flow rate of 3.3 L/min. Such a water jet is efficient at stripping an algal biofilm from a surface up to 2 m away, but the effective area stripped of biofilm is small. Therefore, to cover the entire surface of the carriers, the water jet must be continuously moved.

In some embodiments, the water jet is continuously moved following two actions, a spinning action and a translation action.

The spinning action can be created hydraulically with a self-rotating nozzle, also called a turbo-nozzle. This is a device known in the art available from several suppliers including Spraying Systems Cie, Suttner, Schlick, Lechler, etc. These nozzles are typically used in tank cleaning or pressure washer applications. The preferred nozzles have a jet that is rotating at a speed of several hundreds of revolutions per minute (RPM) around a vertical axis, at a low angle to the vertical, typically 8 to 15 degrees (this corresponds to a full cone angle of 16 to 30 degrees). Suttner turbo-nozzle ST-357 with a full cone angle of 20° is used to illustrate the invention below.

The sum of the rotating nozzle cone angle (8° to) 15° and carrier surface inclination to vertical (3° to 7°)determine the net impact angle on the surface of the carrier. According to the invention, this net impact angle is always smaller that 30 degrees to the vertical, which means that the jet has a significant downward component to drives the dislodged algae through the bottom openings and to the floor of the reactor.

The translation action involves moving an assembly of one or several nozzles over a carrier bed so that substantially all the carrier surfaces is exposed to the water jets of the rotating and laterally moving nozzles. Translation velocity ranges between 0.5-35.0 m/min, most preferably between 1-10 m/min.

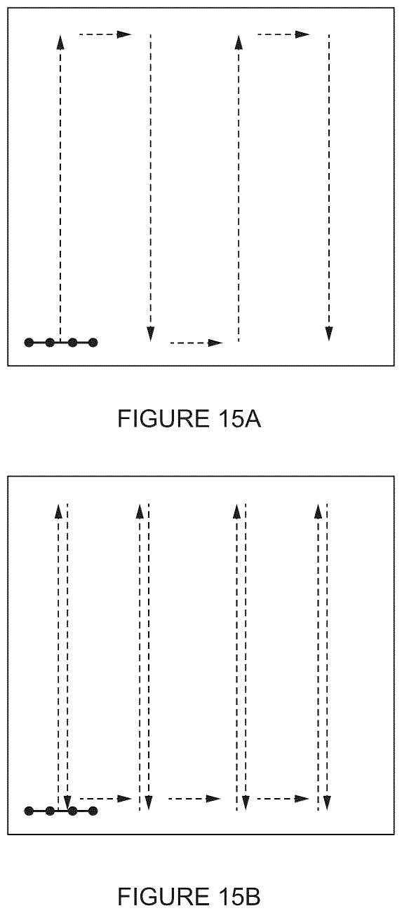

Rotating nozzles are mounted vertically side-by-side on a manifold and moved across the carrier bed. The simplest arrangement is to have a manifold extending for the full width of a carrier bed, but this requires a large number of nozzles and a high harvesting flow rate. As an alternative, FIGS. 15A and 15B show a short manifold containing 4 nozzles (for illustration) which is indexed in the other horizontal direction after completing a pass. In FIG. 15A, indexing is done after a single pass and in FIG. 15B after a double pass (out and back in). Another alternative would be to have an array of manifolds, spread across the carrier bed, with each manifold isolated from the harvesting water flow by a valve. Each manifold could then be activated by opening the valve leading to that manifold for the duration of one or several passes across the carrier bed, after which that valve would close, and the next manifold valve would open. This arrangement requires more nozzles across the entire width of the bed but maintains the lower flow requirements. The valves for each manifold and the movement of the manifolds across the bed would be controlled by a harvest controller to ensure the desired order, duration of harvesting, number of passes, and interval between harvesting events can be optimized.

The jet of a stationary turbo-nozzle traces a circle or a ring on a horizontal plane. The diameter of the ring is proportional to the distance from the nozzle tip. For example, a nozzle with a full cone of 20° forms a 0.35 m ring, 1.0 m from the nozzle tip. Depending on how well the jet maintains its integrity as it flows down, there would not be cleaning/stripping effect inside or outside that ring. The extent of surface coverage is thus dependent on turbo-nozzles spacing to create overlap and on the translation movement.

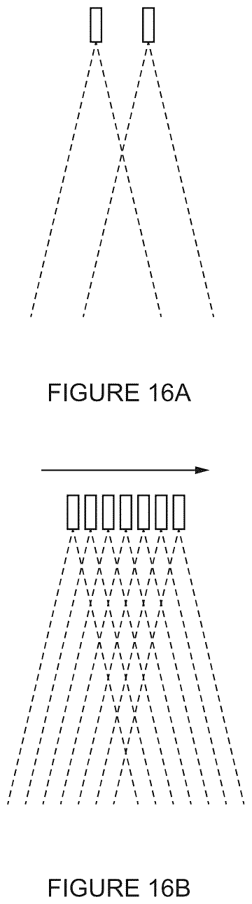

FIG. 16A illustrates how surface coverage is increased by overlapping in the manifold direction. For example, two nozzles with full cone of 20° (shown as broken line) and set 0.20 m apart start overlapping 0.36 m from the nozzle tips and overlap by 76% at 1.0 m from the tips. The overlap may be between 25-100%.

In the direction of translation, the cone slowly progresses as the turbo-nozzle moves, creating significant overlap in the movement direction (FIG. 16B).

### Experimental Validation

Validation experiments were done with the alga Chlorella vulgaris obtained

from the Canadian Phycology Culture Centre (Waterloo, Ontario). The algae were grown using Miracle GRO 24-8-16 at a concentration of 200 mg/L as a source of nutrients dissolved in tap water from the city of Burlington, Ontario.

Experiments were conducted at pilot scale with a carrier bed of 14.4 m² of biofilm surface area covering 1.91 m² footprint. The bed consisted of 144 cones suspended from cables in a 12×12 matrix. Each cone was 50 cm long with bottom diameter of 11.6 cm and made of polyethylene non-woven fabric.

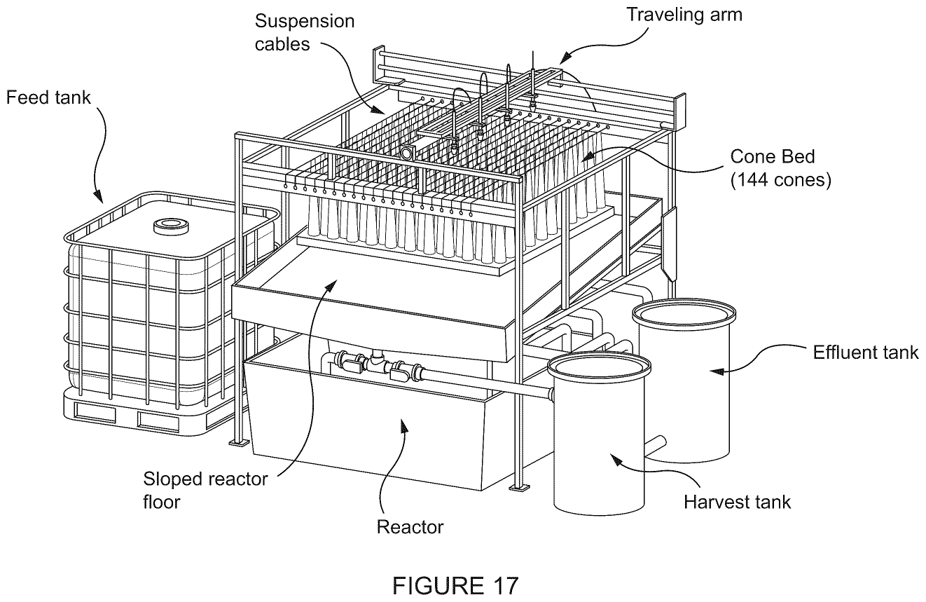

The pilot reactor shown in FIG. 17 was used to grow algae with repeated cycles of irrigation and harvesting. The cone bed was suspended over a sloped floor. The pilot consisted of a feed tank, a reactor tank with a working volume of 500L located underneath the sloped floor and an overflow into an effluent tank. Feed was added to the reactor using a pump at a rate of 100 L/d. Irrigation nozzles creating fine droplets were mounted on a traveling arm moving over the bed every 5 min and were used to irrigate the cone bed with reactor water at a rate of 760 mm/d. Given the high recirculation flow rate, the reactor and the cone bed were considered a CSTR (completely stirred tank reactor). Photosynthetically active radiation (PAR) was generated with an SC Infinity IONFRAME EVO 10 (not shown) which was capable of generating a photon intensity of 1,000 μmol/m^{2/}s at the top of the carriers on a continuous basis.

A harvest was initiated by stopping the recirculation pump and diverting the water collected on the reactor floor to a harvest tank.

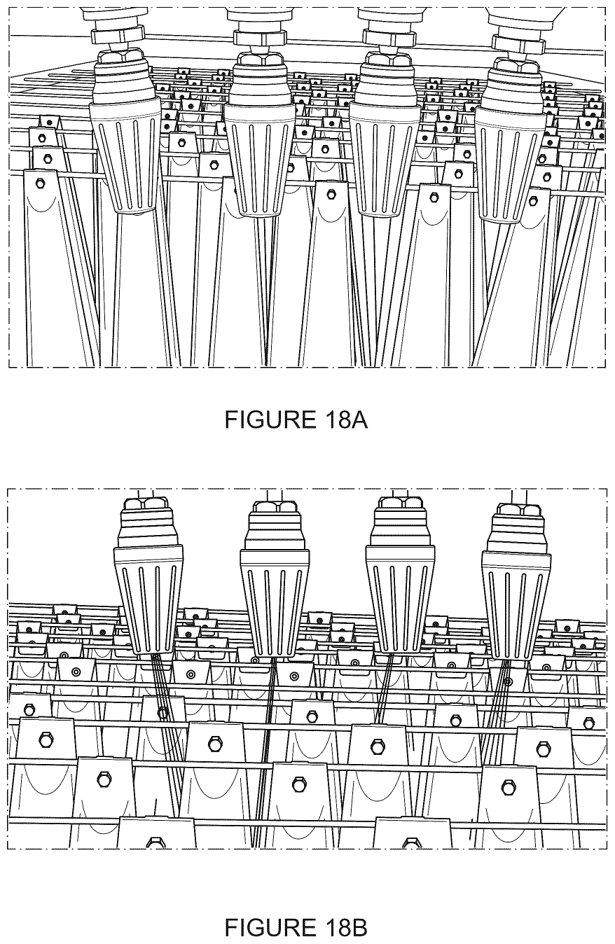

FIG. 18A shows an assembly of 4 turbo-nozzles (Suttner model ST-357, orifice #6, 20° cone) mounted on a manifold 20 cm above the top of the carriers and at a spacing of 20 cm, centre to centre. Adjacent turbo-nozzles therefore provided a spraying overlap of 23% at the bottom of the cones (70 cm), in the direction of the manifold. FIG. 18B shows the nozzles in action shooting solid jets of water down on an algae carrier bed. Each nozzle produced a flow rate of 3.3 L/min at a pressure of 5 bar. FIGS. 18A and 18B were drawn from photographs and are therefor perspective views. The turbo-nozzles appear to be angled towards each other but were actually parallel to each other.

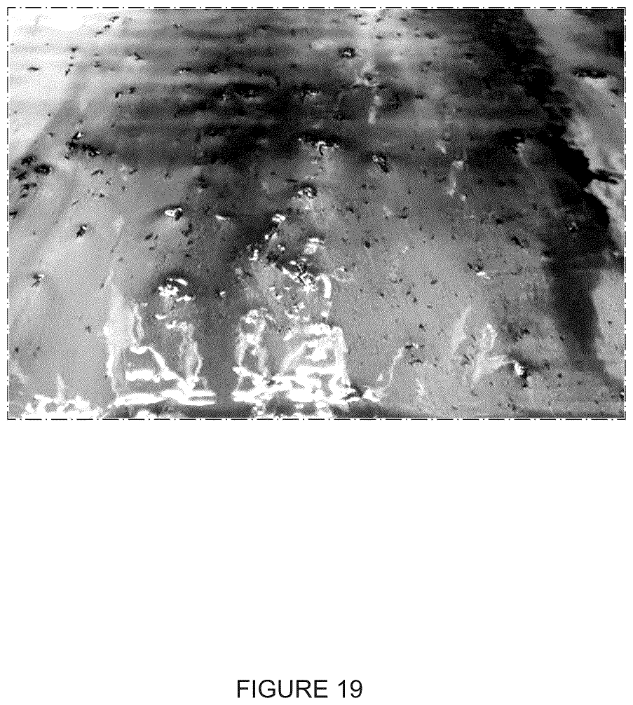

FIG. 19 shows the reactor floor during a harvesting event. Algae agglomerates or blobs are seen flowing down the inclined surface.

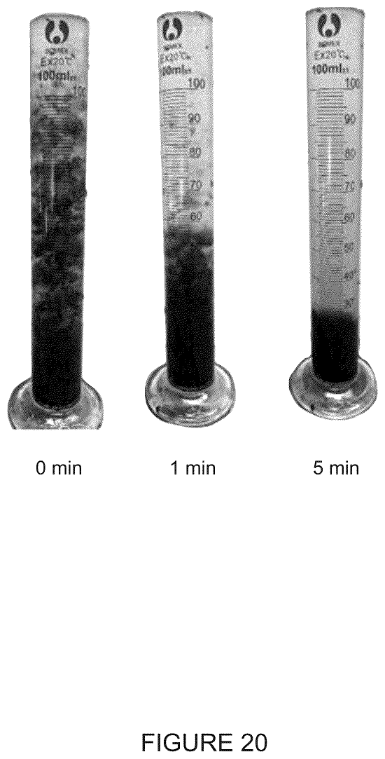

FIG. 20 shows the results of a settling test. Harvesting water was gently blended and poured into a graduated cylinder. The algae readily settled to occupy less than 20% of the volume within 5 minutes (i.e., a concentration factor of 5).

The table below shows the results of 6 harvesting events over a two-month period. All were done following the method described above, but with a variable number of turbo-nozzles and passes as indicated in column 2. The growth period varied between 7 and 12 days. The volume of harvest collected ranged between 105 to 180 liters, or 7 to 12.8 L/m² of carrier. The harvest water contained between 2.5 to 7.8 g/L of algae before any concentration by settling. The mass of algae harvested ranged between 22.7 to 54.3 g/m² of carrier. Finally, the last column shows areal production ranging between 17.1 to 37.2 g/m²/d.

| | Turbo- | | Volume of | | | | |
|---|---|---|---|---|---|---|---|
| | nozzles/ | Growth | Harvest | Harvest | Harvest Water | TS per m² | Aerial |
| | Passes | Period | Water | volume | Concentration | Carrier | Production |
| Date | # | d | L | L/m² | g/L | g/m² | g/m²/d |
| 2024 Apr. 13 | 2/7 | 12.0 | 130 | 9.0 | 3.1 | 28.1 | 17.6 |
| 2024 Apr. 23 | 2/7 | 10.0 | 110 | 7.6 | 3.0 | 22.7 | 17.1 |
| 2024 May 4 | 3/5 | 10.0 | 145 | 10.1 | 3.7 | 37.0 | 27.8 |
| 2024 May 13 | 3/5 | 9.0 | 180 | 12.8 | 2.5 | 31.7 | 26.5 |
| 2024 May 24 | 4/4 | 11.0 | 105 | 7.3 | 3.8 | 28.0 | 19.1 |
| 2024 Jun. 4 | 4/4 | 11.0 | 100 | 7.0 | 7.8 | 54.3 | 37.2 |
| 2024 Jun. 11 | 4/4 | 7.0 | 110 | 7.6 | 3.1 | 23.6 | 25.3 |

### Plant Design

The following paragraphs describe a design for a large-scale algae growing plant. In summary, an algae growing plant can be located outdoors or inside of a greenhouse or other building. Irrigation may be provided with a fixed network of sprinklers. Carriers are optionally fabric cones suspended from cables. A floor below a carrier bed can have a small slope to allow water to flow while retaining dislodged algae on the floor. Harvesting can include two functions, stripping algae from the carriers and recovering the dislodged algae from the floor.

Operation of the plant includes growth and harvesting cycles. At least some species of algae grow better when the growth cycle includes a dark period, and dark periods may also be desirable due to the presence of nighttime in naturally illuminated plants and/or variations in electricity rates in enclosed plants. Although photosynthesis can stop during dark periods, a growth cycle may be considered continuous since reproduction and some biomass accumulation can continue in the dark. Irrigation optionally continues at a reduced rate during dark periods to support the reduced growth rate and/or to avoid drying out the algae.

The growth and harvesting cycles may partially or completely overlap in time. For example, algae stripping and recovery may run substantially continuously during a growth cycle, or during particular parts of a growth cycle such as during dark period. The algae may be harvested from sequential fractions of the carrier bed that cumulatively make up substantially all of the carrier bed. In this way, harvesting continues for extended periods of time but a particular area of the carrier bed is harvested, for example, for less than 5 minutes once every 3 to 10 days. Dislodged algae may sit on a floor below the carriers until recovered, for example by scraping, pushing, blowing or vacuuming. In some embodiments, algae is recovered from the floor at a concentration over 1 wt %, for example 1-50 g/L.

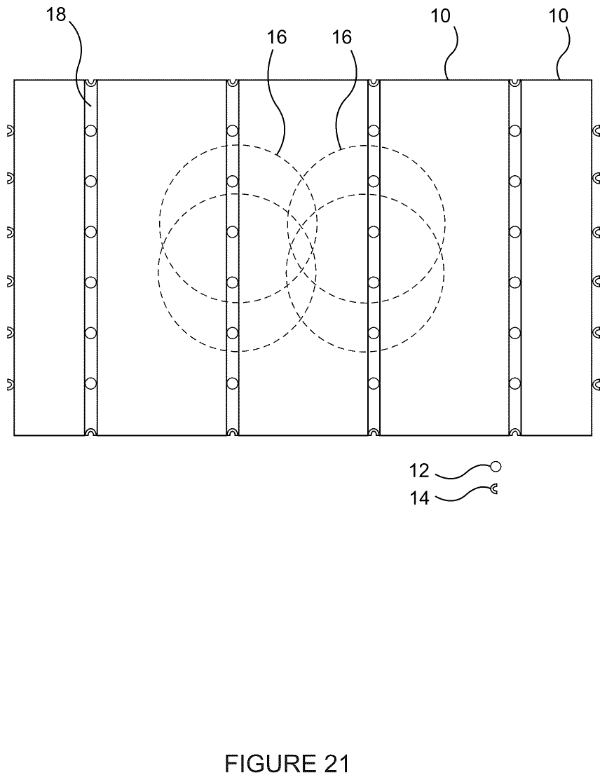

Referring to FIG. 21, a series of carrier beds 10 (shown by their peripheries in FIG. 21) may have a series of sprinklers for irrigation spaced along its length. In the example shown, the sprinklers include 360 degree coverage sprinklers 12 and 180 degree coverage sprinklers 14. 180 degree coverage sprinklers 14 may be spaced along the long sides of a carrier bed 10. 360 degree coverage sprinklers 12 may be placed between the long sides of two adjacent carrier beds 10. Preferably, enough sprinklers 12, 14 are used to provide substantially even irrigation with significant overlap. Spray patterns 16 from four sprinklers 12, 14 are shown in FIG. 21 to give an example of spray pattern overlap but typically all of the sprinklers 12, 14 spray water simultaneously. Optionally, adjacent carrier beds 10 can be separated by a walkway to facilitate servicing the sprinklers 12. The width of a carrier bed 10 may be, for example, 5-12 m.

The sprinklers 12, 14 may be standard agricultural irrigation sprinklers, for example low energy precise application (LEPA) sprinklers. These sprinklers produce uniform rain-like droplets. Each sprinkler may be equipped with a pressure regulator to even out flow between sprinklers. The sprinklers may have a low operating pressure, for example 0.4 to 1.4 bar. Each sprinkler may have a large coverage, for example a throw diameter of 10-15 m, or about 10-12 m when used at a pressure below 0.7 bar. Variable orifice sizes of up to 10 mm can provide solids tolerance. The sprinklers may deliver, for example, up to 1,700 mm/d at 0.7 bar in continuous operation.

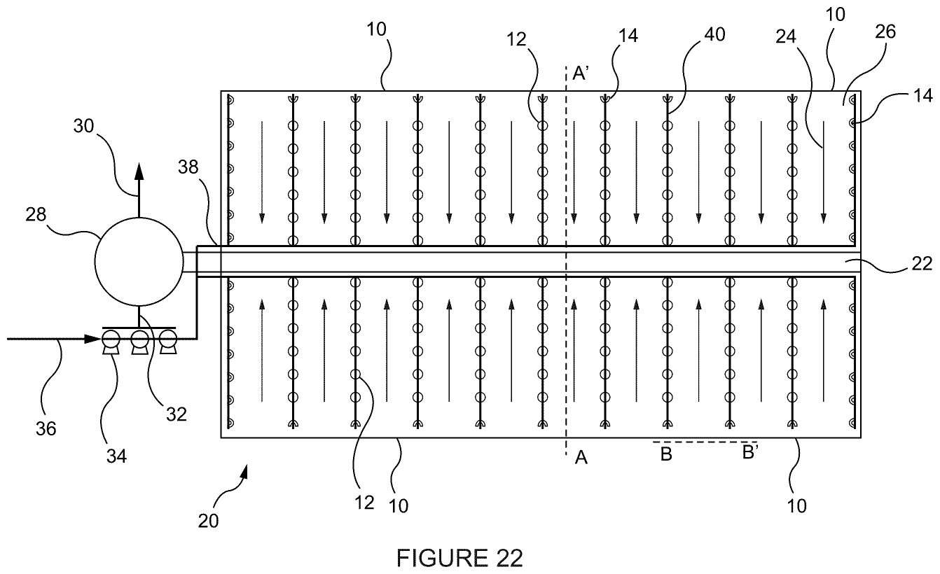

FIG. 22 shows a plant 20 having a plurality of 25 m by 10 m carrier beds 10. The ten carrier beds 10 cumulatively provide 5000 m² of plan view area and may produce 75 tonnes/year of algae. The carrier beds 10 are placed side by side in rows. The ends of two rows of carrier beds 10 are separated by an irrigation channel 22.

Each carrier bed has floor 26 sloped towards the channel 22. The channel 22 is sloped towards a reactor tank 28.

Influent 36, for example wastewater to be treated, is fed by irrigation pumps 34 to irrigation headers 38. The irrigation headers 38 feed irrigation laterals 40, which supply water to the sprinklers 12, 14. Water sprayed from the sprinklers contacts and flows down the carriers (not shown) of the carrier beds 10 and falls to the floor 26. Irrigation water flows in a direction 24 down the sloped floor 26 to the channel 22. The irrigation water in the channel 22 flows towards, and drains into, the reactor tank 28. Some of the irrigation water is released from the tank 28 as a treated effluent 30. Some of the irrigation water is recirculated 32 back to the pumps 34 to be reused for irrigation.

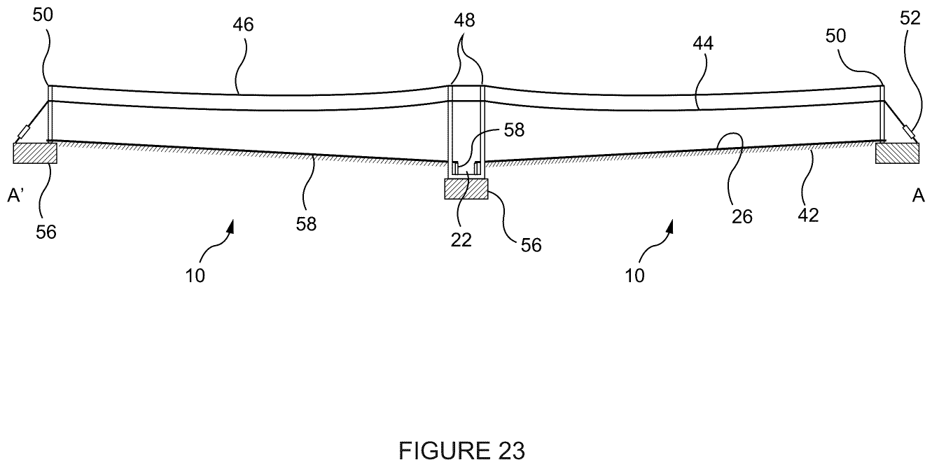

FIG. 23 shows a view through line A′-A of FIG. 22. A series of end gantries 50 and channel gantries 48 are placed along the ends of each carrier bed 10. Multiple gantries 48, 50 spaced across the width of each carrier bed 10 support carrier cables 44. Gantries 48, 50 between adjacent carrier beds, or at the outside of peripheral carrier beds 10, support sprinkler cables 46. The gantries 48, 50 are fixed in concrete anchors 56. End gantries 50 are held up with cable tensioners 52.

Cables 44, 46 are optionally deployed without intermediate supports between the gantries 48, 50. In some embodiments, the cables 44, 46 are made of steel with a diameter of 10-12 mm. The carrier load may be about 8 kg/m. Assuming a permissible sag of 0.5 m, the carrier bed 10 and cables may have a length of, for example, 10-30 m.

A tank to house the carrier beds 10 is not required. Instead, the floor 26 may be provided by a geomembrane 58 placed on sloped earth 42. A geomembrane 58 may also line a channel 22 excavated in the earth. The floor 26 may have a slope of 1-3 degrees, optionally about 2 degrees, towards the channel 22.

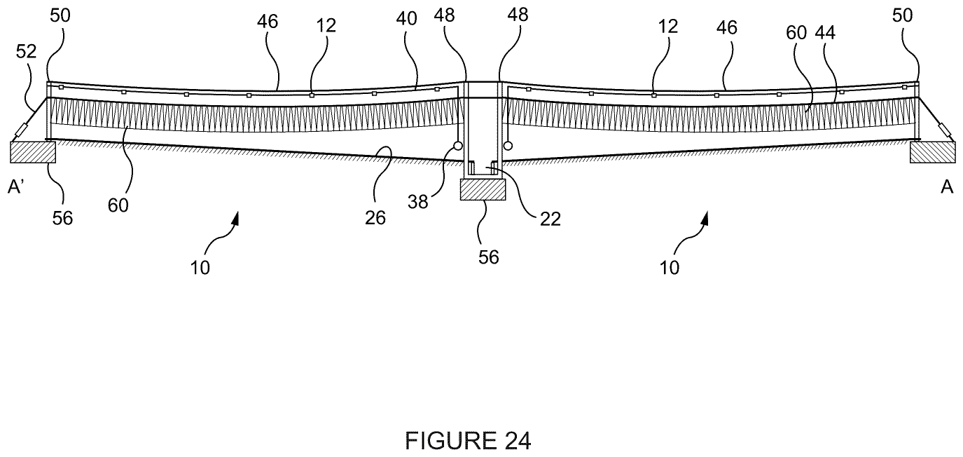

FIG. 24 shows a view through line A′-A of FIG. 22 with additional equipment added. Each carrier cable 44 supports a plurality of suspended carriers 60. In this example, the carriers 60 are fabric cones about 0.1 to 5.0 m tall or 1 to 2 m tall, for example about 1.5 m tall. The sprinkler cables 46 support irrigation laterals 40, which in turn support sprinklers 12, 14. The Irrigation headers 38 to feed water are suspended from the channel gantries 48 so as to be above the floor 26.

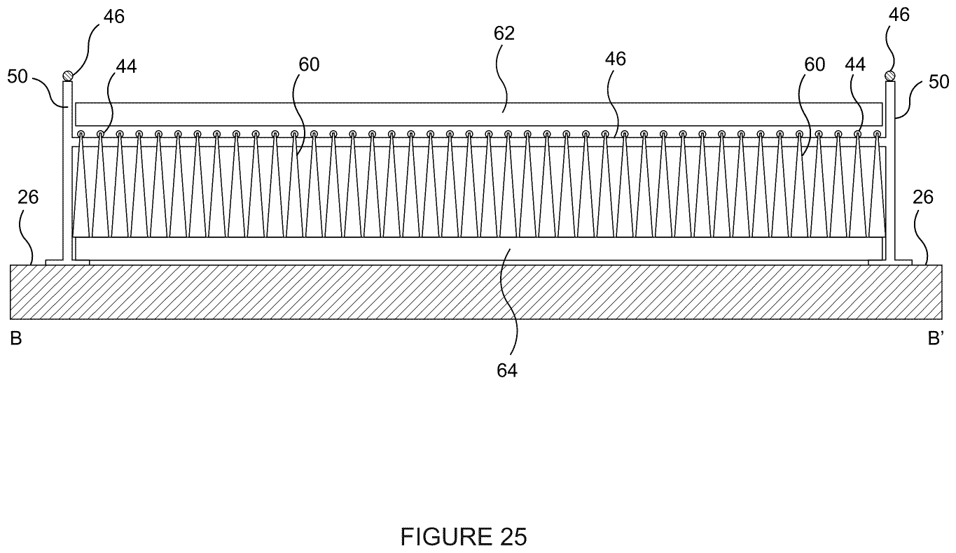

FIG. 25 shows another view through line B-B′ of FIG. 22. Sprinkler cables 46 are located above the carrier cables 44. The space between the sprinkler cables 46 and the carrier cables 44 provides a notional algae stripping volume 62 above the carriers 60 and below the sprinklers 12, 14 and extending horizontally across the plan view area of the carrier beds 10. The algae stripping volume 62 may have a depth of 0.2 m to 1 m, for example about 0.5 m. The algae stripping volume 62 provides a space for the movement of an algae stripping device.

The space between the carriers 60 and the floor 26 and extending horizontally across the plan view area of the carrier beds 10 provides a notional algae recovery volume 64. The algae recovery volume 64 may have a depth of 0.2 m to 1 m, for example about 0.5 m. The algae recovery volume 62 provides a space for the movement of an algae recovery device.

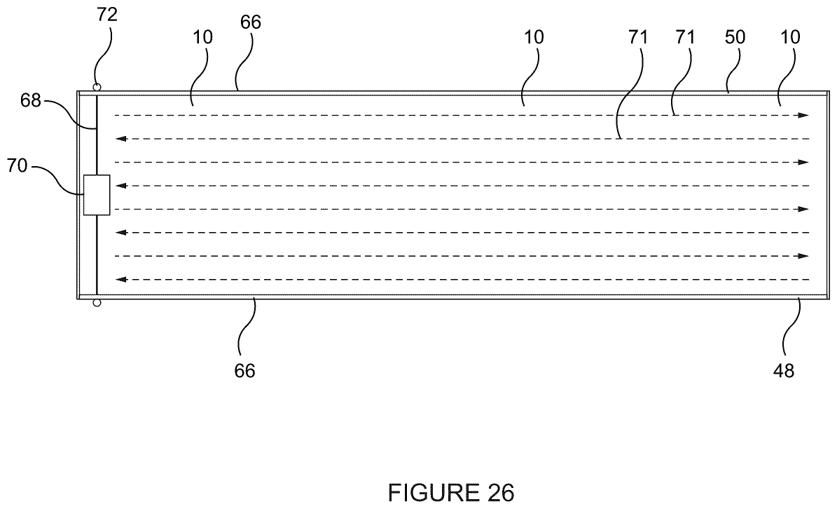

FIG. 26 shows a general scheme for operating an algae stripping device within the algae stripping volume or an algae recovery device within the algae recovery volume over a row of carrier beds, for example one half of the plant 20 of FIG. 22. Rails 66 attached to the gantries 48, 50 support a bridge 68 that travels on the rails 66 back and forth across the row of carrier beds 10. In some embodiments, the bridge 68 includes a cable or beam. The bridge 68 may roll with wheels 72 on the rails 66. A device, for example a manifold, carriage or cart 70 can move and/or be indexed to different lateral positions on the bridge 68. In some embodiments a cart 70 moves, optionally using a powered crawler within the cart 70, along a cable or rigid member of the bridge 68. In other embodiments, a cart 70 is fixed to a cable and the cable moves relative to the bridge 68. In either case, the bridge 68 can translate back and forth across one or more carrier beds 10 along the rails 66 while the cart 70 translates incrementally in a direction from one rail 66 to another. The cart 70 can thereby follow a pattern 71 shown schematically by dashed lines in FIG. 26. In this way, water can be sprayed from the cart 70 over substantially the entire plan view area of one or more carrier beds 10.

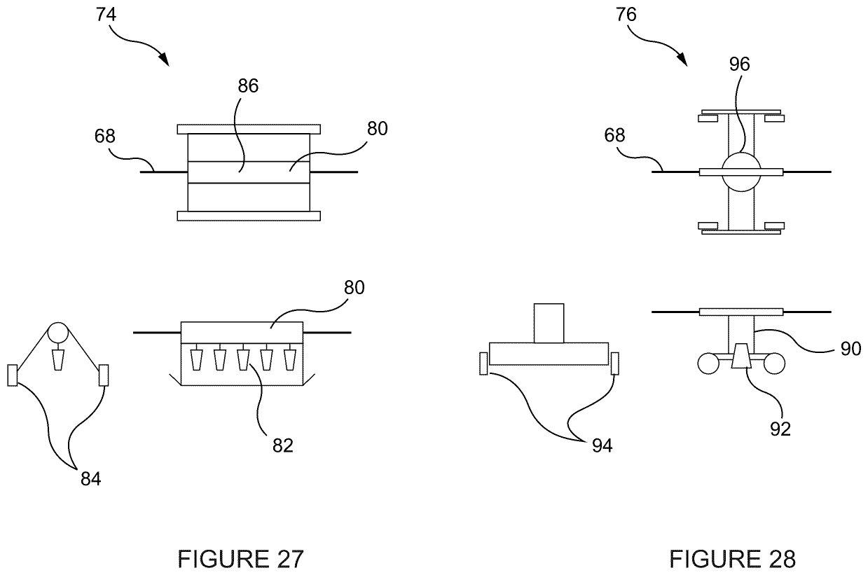

FIG. 27 shows an orthographic projection of a stripping cart 74. The stripping cart 74 has a manifold 80 supported by the cable 68. The manifold 80 distributes pressurized feed water 86 to a plurality of self-rotating water jet nozzles 82. Skates 84 prevent the stripping cart 74 from falling between adjacent pairs of sprinkler cables 46.

In an example, a stripping cart 74 of about 50 cm by 50 cm by 30 cm is attached to a feed water pipe delivering about 12 L/minute of water to a set of rotating nozzles 82. The stripping cart 74 strips a band about 50 cm wide while advancing at about 2 cm/s. The cart strips algae from the carriers 60 at a rate of about 36 square meters of plan view area per hour.

FIG. 28 shows an orthographic projection of a recovery cart 76. The recovery cart 76 has a suction head 92 attached to a vacuum pump 90. Algae vacuumed up by the recovery cart 76 is removed through a product hose 96, which may also include a power supply cable for the vacuum pump 90. Although the recovery cart 76 is supported by cable 68, it may have wheels 94 to allow it to travel on the floor 26.

In an example, a recovery cart 76 of about 50 cm by 50 cm by 30 cm is attached to a suction pipe extracting about 15-20 L/minute of water containing algae from the floor. The recovery cart 76 recovers a band about 50 cm wide while advancing at about 2 cm/s. The recovery cart 76 recovers algae from the floor 26 at a rate of about 36 square meters of plan view area per hour.

Alternatively, a recovery robot may travel along the floor, for example on wheels. The recovery robot may be powered by an on-board battery or an electrical cable. The recovery robot may follow autonomous programming or travel by reference to a wire in the floor or suspended above the recovery robot and below the bottom of the carriers. The recovery robot may scrape, push or blow the algae on the floor to a collection trough. Alternatively, the recovery robot may carry the product pipe as described for the recovery cart 76 and vacuum algae from the floor.

In some embodiments, the floor 26 may have a rough surface, for example

with a Manning coefficient of 0.01 to 0.03, for example about 0.02. In an example, during the day irrigation water is applied at 1,600 mm/d and provides a flow of 26-28 L/min from a 1 m wide by 25 m long strip of floor 26. A film of water on the floor is about 3 mm deep with a velocity of 15 cm/s. During the night, irrigation water is applied at 100 mm/d and provides a flow of 0.7 L/min from the 1 m wide by 25 m long strip of floor. A film of water on the floor is about 0.6 mm deep with a velocity of 5 cm/s. A stripping cart 74 passing across the strip of floor (i.e. perpendicular to the sloping direction of the floor) in the night may add 10 L/min to the flow from the strip, which remains below the daytime flow. The combination of low floor slope, roughness and water velocity is insufficient to entrain algae in the water flowing on the floor. At least some, optionally most, or at least 75% or substantially all of the algae can therefore be recovered from the floor by the recovery cart 76 rather than being washed into the channel 22.

The stripping cart 74 and the recovery cart 76 may complete the pattern 71 in 3-10 days, for example 7 days, optionally while moving only at night. The recovery cart 76 may lag behind the stripping cart 74 by a few minutes to allow stripping water to dissipate and increase the algae concentration on the floor 26 before the algae is recovered. With a harvest for a particular square meter of carrier bed 10 once every 7 days, and an algae production of 40 g/m^{2/}d, stripped algae at a concentration of 2 wt % covers the floor 26 to a thickness of about 10 mm until recovered. Optionally, increasing the lag time may allow the stripped algae to be recovered at a higher concentration due to settling of the algae and some supernatant water flowing to channel 22.

Optionally, the floor 26 may be covered with a mesh material to retain algae while allowing irrigation water to flow to the channel 22. The mesh inhibits large blobs of algae from being entrained by the flow of water down the slope of the floor 26. The mesh may create a large number of mini retention pools to equalize flow and allow algae settling. However, the mesh does not interfere with the operation of the algae recovery cart 76.

The plant 10 may have a height of 5.8 m from the downstream end of the channel 22 to the sprinkler cable 46. The sprinklers 12, 14 may spray water upwards by an additional 0.9 m. Optionally, some or all of the channel 22 or the floor 26 may be provided below grade to reduce the height of the plant 10 above ground.

In a design example, a plant 10 producing 75 tonnes of algae per year (at 40 g/m2/day) can remove about 6,000 kg of nitrogen and 1,000 kg of phosphorous per year from wastewater. The plant captures 150 tonnes/year of CO₂ from the air. The plan view area of the plant 10 is 100 m by 52 m. Irrigation water (i.e. wastewater) is applied at 1,600 mm/d for 12 hours during the daytime and at 100 mm/d for 12 hours during the nighttime. Algae is harvested during the nighttime by hydraulic stripping and slurry pumping to recover the algae from the floor 26. Algae is recovered as a 2 wt % slurry at a flow rate of 17 L/min for 10 hours per day. Energy consumed is about 100,000 kWh/year (95% for irrigation pumping), or about 1.4 kWh/kg algae produced.

Parameters, of the end points of ranges, given in this specification are merely examples and may be varied, for example, by plus or minus 50%.

We claim:

1. A method for dislodging algae from a carrier comprising the steps of,
directing a water jet at a carrier, wherein the water jet is emitted from a nozzle and travels through air to reach the carrier,
rotating the nozzle about an axis oblique to the water jet; and,
translating the nozzle in a direction oblique to the axis.

2. The method of claim 1 wherein the carrier has a vertical or nearly vertical surface for growing the algae.

3. The method of claim 1 wherein the water jet impacts the surface of the carrier at an angle of 45 degrees or less, 30 degrees or less or 20 degrees or less.

4. The method of any of claim 1 comprising dislodging algae from a plurality of carriers are arranged in a pattern in plan view.

5. The method of claim 1 wherein the nozzle is above the carrier.

6. The method of claim 1 wherein the nozzle axis is generally vertical.

7. The method of claim 1 wherein the translation is generally horizontal.

8. The method of claim 1 wherein a plurality of water jets are emitted from a plurality of nozzles which translate collectively.

9. The method of claim 1 wherein a plurality of water jets are emitted from a plurality of nozzles in patterns that overlap each other.

10. The method of claim 1 wherein the translation is according to a pattern including movement in two or more directions.

11. The method of claim 1 wherein a moving harvesting device carrying one or more of the nozzles sprays water at a portion of a carrier bed such that harvesting of the entire bed is distributed over time, for example 3-10 days.

12. The method of claim 1 comprising collecting dislodged algae on a floor below the carrier and recovering the algae from the floor.

13. The method of claim 12 wherein the floor is sloped.

14. The method of claim 13 wherein the translation has a primary direction that is oblique to a slope direction of the floor.

15. The method of claim 12 wherein the algae is recovered from the floor in a slurry or at a concentration of 1-50 g/L.

16. The method of claim 12 comprising recovering the algae from the floor by vacuuming or by moving the algae across the floor for example by pushing, scraping or blowing.

17. The method of claim 12 wherein the algae is recovered using a moving collection device.

18. The method of claim 1 wherein the volume of water used to dislodge the algae is 2-10 L/m2 of carrier surface area.

19. A device to harvest algae growing on a bed of stationary carriers the device comprising an assembly of rotating nozzles mounted on a carriage above the carrier bed wherein the carriage translates above the carrier bed.

20. The device of claim 19 wherein each of the rotating nozzles is adapted to generate a substantially continuous water jet spinning at a rate of 50-500 rpm directed downwards at the carriers in a conical pattern of 15-45 degrees.
